(19) United States
(12) Patent Application Publication
(10) Pub. No.: US 2023/0043556 A1
(43) Pub. Date: Feb. 9, 2023

(54) COMPOSITIONS COMPRISING EXTRACELLULAR VESICLES, SECRETED BIOMOLECULES, AND/OR CONDITIONED MEDIA, AND METHODS OF PRODUCING AND USING THE SAME

(71) Applicant: L'Oreal, Paris (FR)

(72) Inventors: Daniel Christopher Roy, Scotch Plains, NJ (US); Charbel Bouez, Hoboken, NJ (US); Sarah Hubert, Aulnay-Sous-Bois (FR)

(21) Appl. No.: 17/877,814

(22) Filed: Jul. 29, 2022

Related U.S. Application Data
(60) Provisional application No. 63/227,744, filed on Jul. 30, 2021.

(30) Foreign Application Priority Data
Sep. 6, 2021 (FR) 2109323

(51) Int. Cl. A61K 35/36; A61L 27/38; C12N 5/071

(52) U.S. Cl. CPC A61K 35/36; A61L 27/3891; C12N 5/0629; C12N 2502/1323; C12N 2502/094; C12N 2513/00; A61Q 19/00

(57) ABSTRACT

The disclosure relates to novel compositions comprising 1) conditioned media, 2) combinations of secreted biomolecules/organic molecules, and/or 3) secreted extracellular vesicles/exosomes collected from differentiated epithelial cell culture, as well as methods of making and using such compositions.

### CROSS REFERENCE TO RELATED APPLICATIONS

This application claims priority to U.S. Provisional Patent Application No. 63/227,744, filed Jul. 30, 2021, and FR2109323 filed Sep. 6, 2021, both of which are incorporated by reference herein in their entireties.

### TECHNICAL FIELD

The disclosure relates to compositions and methods of making compositions from conditioned media produced by differentiated epithelial cells, and methods of use thereof.

### BACKGROUND

Keratinocytes represent the major cell type of the epidermis, the outermost of the layers of the skin, making up about 90 percent of the cells there. They originate in the deepest layer of the epidermis, the stratum basale and move up to the final barrier layer of the skin, the stratum corneum as they mature. When keratinocytes fully mature they form nucleus-free, flat, and highly keratinized squamous cells in the stratum corneum. The primary function of keratinocytes is the formation of a barrier against environmental damage by heat, UV radiation, water loss, pathogenic bacteria, fungi, parasites, and viruses. Thus, keratinocytes play an important role in a number of physiological processes including aging, protection from sunburns, and modulation of immune responses. Keratinocytes secrete a variety of biomolecules that play a role in these processes. Collection of such biomolecules could prove valuable for the treatment of various conditions.

However, collection of biologically useful amounts and combinations of these biomolecules is hampered by the source of the keratinocytes. In some cases, cell lines are grown as a monolayer or on beads, as opposed to cells grown in three-dimensions (3D). In other cases, cells are embedded in a matrix or suspended in a medium, such as a concentrated medium, that supports the cells in a three-dimensional format. See, e.g., U.S. Pat. No. 8,138,147. Conventional conditioned cell culture media produced by cell-lines grown as a monolayer or on beads is usually discarded. The collection of culture media from tissues with differentiated epithelial layers, such as a basal layer, a stratum spinosum, a granular layer, and a stratum corneum (as found in vivo), has not been disclosed in art. Thus, there are currently no compositions containing the variety of biomolecules found in the conditioned media prepared using the methods described herein.

### SUMMARY

A composition comprising extracellular vesicles and a cosmetic carrier has now been surprisingly and unexpectedly developed. In an embodiment, the extracellular vesicles are derived from a conditioned media collected from differentiated epithelial cells, predecessor keratinocytes, or combinations thereof cultured at an air-liquid interface in a nutrient medium sufficient to meet the nutritional needs required to grow the cells in vitro to form differentiated epithelial cells. In an embodiment, the composition comprises differentiated epithelial cell culture produced conditioned medium-derived extracellular vesicles and a cosmetic carrier, wherein the differentiated epithelial cells are cultured at an air-liquid interface in a nutrient medium sufficient to meet the nutritional needs required to grow the cells in vitro to form differentiated epithelial cells.

In an embodiment, the differentiated epithelial cells are substantially confluent, for example at least 95%, 96%, 97%, 98%, or 99% confluent.

In another embodiment, the differentiated epithelial cells comprise on average at least 2, 3, 4, 5, 6, 7, 8, 9, 10, 11, 12, 13, 14, 15, 16, 17, 18, 19, 20, 21, 22, 23, 24, or 25 living cell layers. For example, the differentiated epithelial cells can comprise on average 1 to 8 cell layers, on average 1 to 2 cell layers, such as 2 living cell layers, or on average 3 to 5 cell layers, such as 3 to 4 living cell layers, or on average 5 to 8 cell layers, such as 6 to 7 living cell layers, or on average 7 to 8 living cell layers, or on average 8 to 9 living cell layers, or on average 8 to 10 living cell layers. One of skill in the art would recognize that the average number of layers can be determined, e.g., by examining a 2D vertical cross-section of the differentiated cells and calculating the average number of cell layers in the cross section. Individual cross sections may differ in their development, thus an average of several cross sections from throughout the sample may also be taken.

The differentiated epithelial cells may form cell layers comprising a basal layer, a stratum spinosum, a granular layer, and/or a stratum corneum.

In an embodiment, the differentiated epithelial cells have a thickness of living cell layers of at least about 2 μm, at least about 3 μm, at least about 4 μm, at least about 5 μm, at least about 6 μm, at least about 7 μm, at least about 8 μm, at least about 9 μm, at least about 10 μm, at least about 11 μm, at least about 12 μm, at least about 13 μm, at least about 14 μm, at least about 15 μm, at least about 16 μm, at least about 17 μm, at least about 18 μm, at least about 19 μm, at least about 20 μm, at least about 21 μm, at least about 22 μm, at least about 23 μm, at least about 24 μm, at least about 25 μm, at least about 26 μm, at least about 27 μm, at least about 28 μm, at least about 29 μm, at least about 30 μm, at least about 31 μm, at least about 32 μm, at least about 33 μm, at least about 34 μm, at least about 35 μm, at least about 36 μm, at least about 37 μm, at least about 38 μm, at least about 39 μm, at least about 40 μm, at least about 41 μm, at least about 42 μm, at least about 43 μm, at least about 44 μm, at least about 45 μm, at least about 46 μm, at least about 47 μm, at least about 48 μm, at least about 49 μm, at least about 50 μm, at least about 51 μm, at least about 52 μm, at least about 53 μm, at least about 54 μm, at least about 55 μm, at least about 56 μm, at least about 57 μm, at least about 58 μm, at least about 59 μm, at least about 60 μm, at least about 61 μm, at least about 62 μm, at least about 63 μm, at least about 64 μm, at least about 65 μm, at least about 66 μm, at least about 67 μm, at least about 68 μm, at least about 69 μm, at least about 70 μm, at least about 71 μm, at least about 72 μm, at least about 73 μm, at least about 74 μm, at least about 75 μm, at least about 76 μm, at least about 77 μm, at least about 78 μm, at least about 79 μm, at least about 80 μm, at least about 81 μm, at least about 82 μm, at least about 83 μm, at least about 84 μm, at least about 85 μm, at least about 86 μm, at least about 87 μm, at least about 88 μm, at least about 89 μm, at least about 90 μm, at least about 91 μm, at least about 92 μm, at least about 93 μm, at least about 94 μm, at least about 95 μm, at least about 96 μm, at least about 97 μm, at least about 98 μm, at least about 99 μm, at least about 100 μm, at least about 101 μm, at least about 102 μm, at least about 103 μm, at least about 104 μm, at least about 105 μm, at least about 106 μm, at least about 107 μm, at least about 108 μm, at least about 109 μm, at least about 110 μm, at least about 111 μm, at least about 112 μm, at least about 113 μm, at least about 114 μm, at least about 115 μm, at least about 116 μm, at least about 117 μm, at least about 118 μm, at least about 119 μm, or at least about 120 μm.

For example, the differentiated epithelial cells may have a thickness of living cell layers of about from 2 μm to about 141 μm, from about 2 μm to about 140 μm, from about 2 μm to about 139 μm, from about 2 μm to about 138 μm, from about 2 μm to about 137 μm, from about 2 μm to about 136 μm, from about 2 μm to about 135 μm, from about 2 μm to about 134 μm, from about 2 μm to about 133 μm, from about 2 μm to about 132 μm, from about 2 μm to about 131 μm, from about 2 μm to about 130 μm, from about 2 μm to about 129 μm, from about 2 μm to about 128 μm, from about 2 μm to about 127 μm, from about 2 μm to about 126 μm, from about 2 μm to about 125 μm, from about 2 μm to about 124 μm, from about 2 μm to about 123 μm, from about 2 μm to about 122 μm, from about 2 μm to about 121 μm, or from about 2 μm to about 120 μm.

For another example, the differentiated epithelial cells may have a thickness of living cell layers of from about 70 μm to about 141 μm, from about 70 μm to about 140 μm, from about 70 μm to about 139 μm, from about 70 μm to about 138 μm, from about 70 μm to about 137 μm, from about 70 μm to about 136 μm, from about 70 μm to about 135 μm, from about 70 μm to about 134 μm, from about 70 μm to about 133 μm, from about 70 μm to about 132 μm, from about 70 μm to about 131 μm, from about 70 μm to about 130 μm, from about 70 μm to about 129 μm, from about 70 μm to about 128 μm, from about 70 μm to about 127 μm, from about 70 μm to about 126 μm, from about 70 μm to about 125 μm, from about 70 μm to about 124 μm, from about 70 μm to about 123 μm, from about 70 μm to about 122 μm, from about 70 μm to about 121 μm, or from about 70 μm to about 120 μm, such as from about 71 μm to about 120 μm, from about 72 μm to about 120 μm, from about 73 μm to about 120 μm, from about 74 μm to about 120 μm, from about 75 μm to about 120 μm, from about 76 μm to about 120 μm, from about 77 μm to about 120 μm, from about 78 μm to about 120 μm, from about 79 μm to about 120 μm, from about 80 μm to about 120 μm, such as from about 81 μm to about 120 μm, from about 82 μm to about 120 μm, from about 83 μm to about 120 μm, from about 84 μm to about 120 μm, from about 85 μm to about 120 μm, from about 86 μm to about 120 μm, from about 87 μm to about 120 μm, from about 88 μm to about 120 μm, from about 89 μm to about 120 μm, or from about 90 μm to about 120 μm.

For another example, the differentiated epithelial cells may have a thickness of living cell layers of from about 2 μm to about 70 μm, from about 2 μm to about 69 μm, from about 2 μm to about 68 μm, from about 2 μm to about 67 μm, from about 2 μm to about 66 μm, from about 2 μm to about 65 μm, from about 2 μm to about 64 μm, from about 2 μm to about 63 μm, from about 2 μm to about 62 μm, from about 2 μm to about 61 μm, from about 2 μm to about 60 μm, from about 2 μm to about 59 μm, from about 2 μm to about 58 μm, from about 2 μm to about 57 μm, from about 2 μm to about 56 μm, from about 2 μm to about 55 μm, from about 2 μm to about 54 μm, from about 2 μm to about 53 μm, from about 2 μm to about 52 μm, from about 2 μm to about 51 μm, from about 2 μm to about 50 μm, from about 2 μm to about 49 μm, from about 2 μm to about 48 μm, from about 2 μm to about 47 μm, from about 2 μm to about 46 μm, from about 2 μm to about 45 μm, from about 2 μm to about 44 μm, from about 2 μm to about 43 μm, from about 2 μm to about 42 μm, from about 2 μm to about 41 μm, from about 2 μm to about 40 μm, from about 2 μm to about 39 μm, from about 2 μm to about 38 μm, from about 2 μm to about 37 μm, from about 2 μm to about 36 μm, from about 2 μm to about 35 μm, from about 2 μm to about 34 μm, from about 2 μm to about 33 μm, from about 2 μm to about 32 μm, from about 2 μm to about 31 μm, from about 2 μm to about 30 μm, from about 2 μm to about 29 μm, from about 2 μm to about 28 μm, from about 2 μm to about 27 μm, from about 2 μm to about 26 μm, from about 2 μm to about 25 μm, from about 2 μm to about 24 μm, from about 2 μm to about 23 μm, from about 2 μm to about 22 μm, from about 2 μm to about 21 μm, from about 2 μm to about 20 μm, from about 2 μm to about 19 μm, from about 2 μm to about 18 μm, from about 2 μm to about 17 μm, from about 2 μm to about 16 μm, from about 2 μm to about 15 μm, from about 2 μm to about 14 μm, from about 2 μm to about 13 μm, from about 2 μm to about 12 μm, from about 2 μm to about 11 μm, from about 2 μm to about 10 μm, from about 2 μm to about 9 μm, from about 2 μm to about 8 μm, from about 2 μm to about 7 μm, from about 2 μm to about 6 μm, from about 2 μm to about 5 μm, from about 2 μm to about 4 μm, or from about 2 μm to about 3 μm.

For another example, the differentiated epithelial cells may have a thickness of living cell layers of from about 3 μm to about 70 μm, from about 4 μm to about 70 μm, from about 5 μm to about 70 μm, from about 6 μm to about 70 μm, from about 7 μm to about 70 μm, from about 8 μm to about 70 μm, from about 9 μm to about 70 μm, from about 10 μm to about 70 μm, from about 11 μm to about 70 μm, from about 12 μm to about 70 μm, from about 13 μm to about 70 μm, from about 14 μm to about 70 μm, from about 15 μm to about 70 μm, from about 16 μm to about 70 μm, from about 17 μm to about 70 μm, from about 18 μm to about 70 μm, from about 19 μm to about 70 μm, from about 20 μm to about 70 μm, from about 21 μm to about 70 μm, from about 22 μm to about 70 μm, from about 23 μm to about 70 μm, from about 24 μm to about 70 μm, from about 25 μm to about 70 μm, from about 26 μm to about 70 μm, from about 27 μm to about 70 μm, from about 28 μm to about 70 μm, from about 29 μm to about 70 μm, from about 30 μm to about 70 μm, from about 31 μm to about 70 μm, from about 32 μm to about 70 μm, from about 33 μm to about 70 μm, from about 34 μm to about 70 μm, from about 35 μm to about 70 μm, from about 36 μm to about 70 μm, from about 37 μm to about 70 μm, from about 38 μm to about 70 μm, from about 39 μm to about 70 μm, from about 40 μm to about 70 μm, from about 41 μm to about 70 μm, from about 42 μm to about 70 μm, from about 43 μm to about 70 μm, from about 44 μm to about 70 μm, from about 45 μm to about 70 μm, from about 46 μm to about 70 μm, from about 47 μm to about 70 μm, from about 48 μm to about 70 μm, from about 49 μm to about 70 μm, from about 50 μm to about 70 μm, from about 51 μm to about 70 μm, from about 52 μm to about 70 μm, from about 53 μm to about 70 μm, from about 54 μm to about 70 μm, from about 55 μm to about 70 μm, from about 56 μm to about 70 μm, from about 57 μm to about 70 μm, from about 58 μm to about 70 μm, from about 59 μm to about 70 μm, from about 60 μm to about 70 μm, from about 61 μm to about 70 μm, from about 62 μm to about 70 μm, from about 63 μm to about 70 μm, from about 64 μm to about 70 μm, from about 65 μm to about 70 μm, from about 66 μm to about 70 μm, from about 67 μm to about 70 μm, from about 68 μm to about 70 μm, or from about 69 μm to about 70 μm.

In an embodiment, the differentiated epithelial cells have differentiated over predecessor human keratinocytes.

In some embodiments, the differentiated epithelial cells may be cultured for at least 1, 2, 3, 4, 5, 6, 7, 8, 9, 10, 11, 12, 13 14, 15, 16, 17, 18, 19, 20, 21, 22, 23, 24, 25, 26, 27, 28, 29, 30, 31, 32, 33, 34, or 35 days, such as from between 1 to 35 days, 2 to 34 days, 2 to 33 days, 2 to 32 days, 2 to 31 days, 2 to 30 days, 2 to 29 days, 2 to 28 days, 2 to 27 days, 2 to 26 days, 2 to 25 days, 2 to 24 days, 2 to 23 days, 2 to 22 days, 2 to 21 days, 2 to 20 days, 2 to 19 days, 2 to 18 days, 2 to 17 days, 2 to 16 days, 2 to 15 days, 2 to 14 days, 2 to 13 days, 2 to 12 days, 2 to 11 days, 2 to 10 days, 2 to 9 days, 2 to 8 days, 2 to 7 days, 3 to 16 days, 3 to 15 days, 3 to 14 days, 3 to 13 days, 3 to 12 days, 3 to 11 days, 3 to 10 days, 3 to 9 days, 3 to 8 days, 3 to 7 days, 4 to 16 days, 4 to 15 days, 4 to 14 days, 4 to 13 days, 4 to 12 days, 4 to 11 days, 4 to 10 days, 4 to 9 days, 4 to 8 days, 4 to 7 days, 5 to 16 days, 5 to 15 days, 5 to 14 days, 5 to 13 days, 5 to 12 days, 5 to 11 days, 5 to 10 days, 5 to 9 days, 5 to 8 days, or 5 to 7 days.

The differentiated epithelial cells may be cultured on a porous substrate. For example, the differentiated epithelial cells may be incubated for at least 3 hours. The differentiated epithelial cells may be incubated at about 36° C. to about 38° C. The differentiated epithelial cells may be incubated at about 4%-6% CO2. In an embodiment, the differentiated epithelial cells are incubated at about 10% to about 100% humidity, preferably at about 20% to about 99%, preferably at about 30% to about 99%, preferably at about 40% to about 100% humidity, such as at about 50% to about 99%, such as at about 60% to about 98%, preferably at about 70% to about 97%, preferably at about 45% to about 80%, more preferably at about 50% to about 70%, more preferably at about 55% to about 65%, more preferably at about 92% to about 97%, or more preferably at about 94% to about 96%.

In some embodiments, the conditioned medium comprising the extracellular vesicles is concentrated, filtered, and/or purified prior to combining the extracellular vesicles with a carrier to form the composition. The extracellular vesicles may be isolated from the conditioned media prior to combining the extracellular vesicles with a carrier to form the composition. The extracellular vesicles may be lysed prior to combination with a carrier.

In an embodiment, the nutrient medium is a chemically defined medium. The differentiated epithelial cells may be cultured in batch culture.

In an embodiment, the extracellular vesicles comprise large extracellular vesicles having a diameter of more than 150 nm and small extracellular vesicles having a diameter 150 nm or less, and wherein the ratio of the small extracellular vesicles to the large extracellular vesicles is no greater than about 30.5:1, no greater than about 30:1, no greater than about 29.5:1, no greater than about 29:1, no greater than about 28.5:1, no greater than about 28:1, no greater than about 27.5:1, no greater than about 27:1, no greater than about 26.5:1, no greater than about 26:1, no greater than about 25.5:1, no greater than about 25:1, no greater than about 24.5:1, no greater than about 24:1, no greater than about 23.5:1, no greater than about 23:1, no greater than about 22.5:1, no greater than about 22:1, no greater than about 21.5:1, no greater than about 21:1, no greater than about 20.5:1, no greater than about 20:1, no greater than about 19.5:1, no greater than about 19:1, no greater than about 18.5:1, no greater than about 18:1, no greater than about 17.5:1, no greater than about 17:1, no greater than about 16.5:1, no greater than about 16:1, no greater than about 15.5:1, no greater than about 15:1, no greater than about 14.5:1, no greater than about 14:1, no greater than about 13.5:1, no greater than about 13:1, no greater than about 12.5:1, no greater than about 12:1, no greater than about 11.5:1, no greater than about 11:1, no greater than about 10.5:1, no greater than about 10:1, no greater than about 9.5:1, no greater than about 9:1, no greater than about 8.5:1, no greater than about 8:1, no greater than about 7.5:1, no greater than about 7:1, no greater than about 6.5:1, no greater than about 6:1, no greater than about 5.5:1, no greater than about 5:1, no greater than about 4.5:1, no greater than about 4:1, no greater than about 3.5:1, no greater than about 3:1, no greater than about 2.5:1, no greater than about 2:1, no greater than about 1.5:1, or no greater than about 1:1. The ratio may be determined by, for example, a) separating the small extracellular vesicles from the conditioned medium, e.g. using a combination of tangential flow filtration (TFF) and size-exclusion chromatography (SEC), then b) detecting the number of small extracellular vesicles, and a) separating the large extracellular vesicles from the conditioned medium by TFF, then b) detecting the number of large extracellular vesicles. The small and large EVs can be separated, then detected, in any order or simultaneously. The separated extracellular vesicles can be detected using any means including, but not limited to, nanoparticle tracking analysis (NTA) and ELISA.

In an embodiment, the extracellular vesicles comprise large extracellular vesicles having a diameter of more than 150 nm and small extracellular vesicles having a diameter 150 nm or less, and wherein the ratio of the small extracellular vesicles to the large extracellular vesicles ranges from about 1:1 to about 30.5:1, from about 1:1 to about 30:1, from about 1:1 to about 29.5:1, from about 1:1 to about 29:1, from about 1:1 to about 28.5:1, from about 1:1 to about 28:1, from about 1:1 to about 27.5:1, from about 1:1 to about 27:1, from about 1:1 to about 26.5:1, from about 1:1 to about 26:1, from about 1:1 to about 25.5:1, from about 1:1 to about 25:1, from about 1:1 to about 24.5:1, from about 1:1 to about 24:1, from about 1:1 to about 23.5:1, from about 1:1 to about 23:1, from about 1:1 to about 22.5, from about 1:1 to about 22:1, from about 1:1 to about 21.5:1, from about 1:1 to about 21:1, from about 1:1 to about 20:1, from about 1:1 to about 19:1, about from 1:1 to about 18:1, from about 1:1 to about 17:1, from about 1:1 to about 16:1, from about 1:1 to about 15:1, from about 1:1 to about 14:1, from about 1:1 to about 13:1, from about 1:1 to about 12:1, from about 1:1 to about 11:1, from about 1:1 to about 10:1, from about 1:1 to about 9:1, from about 1:1 to about 8:1, from about 1:1 to about 7:1, from about 1:1 to about 6:1, from about 1:1 to about 5:1, from about 1:1 to about 4:1, from about 1:1 to about 3:1, from about 1:1 to about 2:1, from about 2:1 to about 20:1, from about 2:1 to about 19:1, from about 2:1 to about 18:1, from about 2:1 to about 17:1, from about 2:1 to about 16:1, from about 2:1 to about 15:1, from about 2:1 to about 14:1, from about 2:1 to about 13:1, from about 2:1 to about 12:1, from about 2:1 to about 11:1, from about 2:1 to about 10:1, from about 2:1 to about 9:1, from about 2:1 to about 8:1, from about 2:1 to about 7:1, from about 2:1 to about 6:1, from about 2:1 to about 5:1, from about 2:1 to about 4:1, from about 2:1 to about 3:1, from about 3:1 to about 20:1, from about 3:1 to about 19:1, from about 3:1 to about 18:1, from about 3:1 to about 17:1, from about 3:1 to about 16:1, from about 3:1 to about 15:1, from about 3:1 to about 14:1, from about 3:1 to about 13:1, from about 3:1 to about 12:1, from about 3:1 to about 11:1, from about 3:1 to about 10:1, from about 3:1 to about 9:1, from about 3:1 to about 8:1, from about 3:1 to about 7:1, from about 3:1 to about 6:1, from about 3:1 to about 5:1, or from about 3:1 to about 4:1 when a) separating the small extracellular vesicles from the conditioned medium, using a combination of tangential flow filtration (TFF) and size-exclusion chromatography (SEC), then b) detecting the number of small extracellular vesicles, and a) separating the large extracellular vesicles from the conditioned medium by TFF, then b) detecting the number of large extracellular vesicles.

In an embodiment, the composition comprises a population of CD9 positive extracellular vesicles wherein the ratio of CD63 to CD81 detected in the population is at least about 0.2:1, from at least about 0.3:1, from at least about 0.4:1, from at least about 0.5:1, from at least about 0.6:1, from at least about 0.7:1, from at least about 0.8:1, from at least about 0.9:1, or from at least about 1:1, or from at least about 1.1:, or from at least about 1.2:1, or from at least about 1.3:1, or from at least about 1.4:1, or from at least about 1.5:1, or from at least about 1.6:1, or from at least about 1.7:1, or from at least about 1.9:1, or from at least about 2:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

In an embodiment, the composition comprises a population of CD9 positive extracellular vesicles wherein the ratio of CD63 to CD81 detected in the population ranges from about 0.2:1 to about 2.8:1, from about 0.2:1 to about 2.7:1, from about 0.3:1 to about 2.6:1, from about 0.3:1 to about 2.5:1, from about 0.4:1 to about 2.4:1, from about 0.4:1 to about 2.3:1, from about 0.5:1 to about 2.2:1, from about 0.6:1 to about 2.1:1, from about 0.6:1 to about 2.0:1, from about 0.6:1 to about 1.9:1, from about 0.7:1 to about 1.8:1, from about 0.7:1 to about 1.7:1, from about 0.8:1 to about 1.6:1, from about 0.8:1 to about 1.5:1, from about 0.9:1 to about 1.4:1, or from about 0.9:1 to about 1.3:1. The ratio may be determined by, for example, detecting CD63 and CD81 on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

In an embodiment, the composition further comprises small molecules, biologics, therapeutic agents, preservatives, or enzymes.

Methods of making a composition comprising extracellular vesicles and a carrier, wherein the extracellular vesicles are derived from a conditioned medium collected from differentiated epithelial cells cultured at an air-liquid interface in a nutrient medium sufficient to meet the nutritional needs required to grow the cells in vitro to form differentiated epithelial cells are also disclosed.

Methods of treatment comprising applying the disclosed compositions to epidermal cells or skin are also disclosed. In an embodiment, a method for promoting fibroblast growth comprising applying the disclosed compositions of any one of claims to skin or epidermal cells is disclosed herein. In an embodiment, a method for increasing keratinocyte growth factor secretion or release comprising applying the disclosed composition to skin is disclosed herein. In an embodiment, a method for inducing keratinocyte growth factor secretion or release comprising applying the disclosed composition to skin is disclosed herein. A method of use comprising applying the composition to skin is also disclosed herein.

It is to be understood that both the foregoing general description and the following detailed description present various embodiments of the disclosure and are intended to provide an overview or framework for understanding the nature and character of the claims.

### BRIEF DESCRIPTION OF THE FIGURES

FIG. 1 shows a stained vertical cross-section of the tissue architecture of a reconstructed human epidermis at day 17 of cell culture.

FIG. 2 demonstrates the expression of EV surface markers on CD9+ comprising small extracellular vesicles collected from differentiated epithelial cell conditioned media at different time points (A-C) versus small extracellular vesicles collected from 2D keratinocytes (D) as measured using enzyme-linked immunosorbent assays. The immunodetection of protein (ratio of absorbance value of detection to background) is normalized per total amount of particles (A-C). Ratio of normalized immunodetection of CD63 to CD81 is compared to extracellular vesicles from 2D keratinocytes (D).

FIG. 3 demonstrates the expression of EV surface markers on large extracellular vesicles from differentiated epithelial cell conditioned media (A-C) and 2D keratinocyte conditioned media (D) as measured using ELISA. The immunodetection of protein (ratio of absorbance value of detection to background) is normalized per total amount of particles.

FIG. 4 demonstrates the expression of internal markers of small extracellular vesicles collected from differentiated epithelial cell conditioned media as measured using enzyme-linked immunosorbent assays. The immunodetection of protein (ratio of absorbance value of detection to background) is normalized per total amount of particles.

FIG. 5 demonstrates the expression of internal markers of large extracellular vesicles collected from differentiated epithelial cell conditioned media as measured using enzyme-linked immunosorbent assays. The immunodetection of protein (ratio of absorbance value of detection to background) is normalized per total amount of particles.

FIGS. 6A-6D demonstrate normal human dermal fibroblast cell growth after exposure to small extracellular vesicles. The small EVs are obtained from two batches of differentiated epithelial cell conditioned media on days 3, 6 and 17 of cell culture (FIGS. 6A-C respectively), and from two batches of two-dimensional keratinocyte cultures (FIG. 6D).

FIGS. 7A-7C demonstrate keratinocyte growth factor (KGF) secretion, as measured using enzyme-linked immunosorbent assays (ELISA), by normal human dermal fibroblast into culture media after exposure of the cells to extracellular vesicles. FIG. 7A demonstrates fibroblast secretion of KGF after exposure to 1_{E}⁸ particles/mL of small extracellular vesicles from the day 3, 6, and 17 differentiated epithelial cell culture media and 2D keratinocyte culture media. FIG. 7B demonstrates fibroblast secretion of KGF after exposure to increasing concentrations of small extracellular vesicles from the day 3 and 6 differential epidermal cell culture media. FIG. 7C demonstrates fibroblast secretion of KGF after exposure to increasing concentrations of small extracellular vesicles from the two-dimensional cell culture media.

It is to be understood that the foregoing and following descriptions are exemplary and explanatory only, and are not intended to be restrictive of any subject matter claimed.

### DETAILED DESCRIPTION

The disclosure relates to novel compositions comprising unique 1) conditioned media, 2) combinations of secreted biomolecules/organic molecules, and/or 3) secreted extracellular vesicles/exosomes collected from differentiated epithelial cell culture, as well as methods of making and using such compositions. The methods of making comprise collecting media at discrete time points from keratinocytes and/or differentiated epithelial cells cultured at the air-liquid interface as the keratinocytes mature into differentiated epithelial cells, and into a reconstructed epidermis. The conditioned media and extracellular vesicles produced by differentiated epithelial cells can be supplemented with additional components (e.g., small molecules, therapeutic agents, etc.) and optionally preserved by a variety of mechanisms including freezing in a liquid state or lyophilizing.

Keratinocytes

Any source of keratinocytes can be used. Keratinocytes are available from individual or from pooled donors, i.e. from more than one donor. Keratinocytes can be obtained from skin isolated from various locations on the human body. Nonlimiting examples of keratinocytes sources include the epidermis of foreskin, the face, the breasts, the abdomen, and the thighs. In various embodiments, the keratinocytes may be human, such as Primary Normal Human Keratinocytes (NHKs) or Primary Normal Human Epidermal Keratinocytes (NHEKs), to reduce the risk of an immune response.

Keratinocytes can be separated from tissues using any means known in the art. In one embodiment, surgical skin samples can be taken from adults and subjected to the 0.25% trypsin dermis/epidermis separation method described in Rheinwald to obtain suspensions of normal human keratinocytes (NHK). See Rheinwald J. G., Green H.: Serial cultivation of strains of human epidermal keratinocytes: The formation of keratinizing colonies from single cells. Cell. 6(3):331-43. 1975.

The cell cultures described herein can be inoculated with cryopreserved (frozen) or proliferating keratinocytes. The extent to which the keratinocytes are grown prior to use in the disclosed cultures may vary.

Pre-Conditioned Media

The “pre-conditioned” cell culture medium may be any nutrient medium, i.e. any cell culture medium which adequately addresses the nutritional needs of the cells being cultured, e.g. any medium that is suitable for in vitro cell culture of human or animal cells or tissues.

In some embodiments, the disclosed cells are cultured in a nutrient medium that is a chemically defined medium, i.e. a cell growth medium in which all of the chemical components are known that is suitable for in vitro cell culture of cells or tissues. Examples of defined media include, but are not limited to Dulbecco's Modified Eagle's Medium (DMEM), Ham's F12, RPMI 1640, Iscove's, McCoy's and other media formulations readily apparent to those skilled in the art, including those found in Methods For Preparation of Media, Supplements and Substrate For Serum-Free Animal Cell Culture Alan R. Liss, New York (1984) and Cell & Tissue Culture: Laboratory Procedures, John Wiley & Sons Ltd., Chichester, England 1996, both of which are incorporated by reference herein in their entirety.

In some embodiments, the disclosed cells are cultured in a nutrient medium that is an undefined medium, i.e. a cell growth medium suitable for in vitro cell culture of cells or tissues in which all of the chemical components are not known.

In certain embodiments, the pre-conditioned media is serum-free and/or animal product-free. Serum-free and animal product-free (sometimes referred to as protein-free) media is commercially available from, among other vendors, LifeTechnologies-GibcoBRL, Rockville, Md.; Sigma-Aldrich, Saint Louis, Mo.; or BioWhittaker, Walkersville, Md.). Exemplary serum-free media include: UltraCULTURE™, UltraDOMA™ and UltraCHO™, from BioWhittaker; Serum-free Hybridoma Medium, CHO Serum-free Medium, and MDCK Serum-free Medium, from Sigma-Aldrich; and Keratinocyte-SFM (KSFM), AIM V® Media, Stem Pro®-34 SFM, Human Endothelial-SFM, Macrophage-SFM, and HepatoZYME-SFM from Life Technologies. Exemplary protein-free media include: UltraDOMA-PF™ from BioWhittaker; Animal Component-free Hybridoma Medium, Serum-free and Protein-free Hybridoma Medium Hybri-Max®, CHO Protein-free Medium, Chemically-defined CHO Medium, and MDCK Protein-free Medium from Sigma-Aldrich; and Defined Keratinocyte-SFM from Life Technologies. The skilled artisan will appreciate that the use of serum-free media for mammalian cell culture is well established, and is described in, among other places, Cold Spring Harbor Conferences on Cell Proliferation, Vol. 9, Sato et al., eds., (1982) Cold Spring Harbor Laboratory, Cold Spring Harbor, N.Y.; Barnes et al., Anal. Biochem. 102, 255 (1980); BioWhittaker 1999/2000 catalog, pp. 42-51; Barnes, Serum-Free Animal Cell Culture, BioTechniques 5(6):534-42; and Freshney, Culture of Animal Cells, 3d ed., Wiley-Liss, New York, N.Y., 1994.

The medium may be supplemented with any ingredients useful or necessary to support the disclosed keratinocyte cell/differentiated epithelial cell tissue culture. Pre-conditioned media ingredients include, but are not limited to, amino-acids (both D and/or L-amino acids) such as glutamine, alanine, arginine, asparagine, cysteine, glutamic acid, glycine, histidine, isoleucine, leucine, lysine, methionine, phenylalanine, proline, serine, threonine, tryptophan, tyrosine, and valine and their derivatives; acid soluble subgroups such as thiamine, ascorbic acid, ferric compounds, ferrous compounds, purines, glutathione and monobasic sodium phosphates.

Additional ingredients include sugars, deoxyribose, ribose, nucleosides, water soluble vitamins, riboflavin, salts, trace metals, lipids, acetate salts, phosphate salts, HEPES, phenol red, pyruvate salts and buffers.

Other ingredients can be selected by those of skill in the art in accordance with his or her particular need.

In an embodiment, the conditioned media may be replaced with pre-conditioned media every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days during cell culture (starting from the day that the keratinocytes are seeded), or any timepoint therebetween.

Three-Dimensional Substrate

In certain embodiments, the three-dimensional cell culture comprises a substrate for the cells to grow on and/or adhere to. A three-dimensional “substrate” or “framework” or “support” or “scaffold” as used herein refers to a three-dimensional material composed of any substance and/or of any three-dimensional shape that (a) allows cells to attach to it (or can be modified to allow cells to attach to it); and (b) allows cells to grow in more than one layer. As disclosed herein, this substrate is inoculated with keratinocytes to form the living three-dimensional tissue.

In an embodiment, the structure of the substrate can be a porous growth substrate, such as a mesh, sponge, membrane, film, filter or hydrogel.

A number of different materials may be used to form the substrate, including but not limited to: non-biodegradable materials, e.g., nylon (polyamides), dacron (polyesters), polystyrene, polypropylene, polyethylene, polyacrylates, polyolefin, polyvinyl compounds (e.g., polyvinylchloride), polycarbonate (PC), polytetrafluorethylene (PTFE; teflon), polysulfone, silicone thermanox (TPX), nitrocellulose, ethylene vinyl acetate, cotton; and biodegradable materials, e.g., polyglycolic acid (PGA), collagen, collagen sponges, cat gut sutures, cellulose, gelatin, dextran, polyalkanoates, etc. Any of these materials may be woven braided, knitted, etc., into a mesh, for example, to form the three-dimensional substrate.

In some embodiments, the substrate can be an ionomer resin film which is optically clear and non-toxic to cells. Suitable non-ionomeric resins such as, by way of example only, polycarbonates, polystyrenes, or polyfluorinated polymers, can be used. In some embodiments, polycarbonate films such as films comprising a 0.33 cm² surface (Nucell, Nucleopore, and Transwell, Costar, France) are used.

In some embodiments, the substrate is gas permeable and/or liquid impermeable, e.g. may be a gas-permeable, liquid impermeable film or membrane. Thickness of the gas permeable membrane will depend on the desired resultant characteristics which may include, but are not limited to, structural integrity, degree of gas permeability, and rate of transfer of gases. The gas permeable membrane may be comprised of one or more membranes known in the art.

In a preferred embodiment, the disclosed cells are grown in multiple layers on a three-dimensional substrate, forming differentiated epithelial cells.

Cell Culture at the Air-Liquid Interface

The differentiated epithelial cells/keratinocyte predecessors are cultured at the “air-liquid interface” (wherein the cultured cells are exposed to air on one surface and nutrient medium on another surface) using any means known in the art. Preferably, the cells are cultured in an environment which enables aseptic processing and handling.

In an embodiment, the cells are incubated for at least 3 hours. The cells can be incubated, for example, at about 36° C. to about 38° C. The cells can also be incubated, for example, at about 4%-6% CO2. The cells can also be incubated, for example, at about 10% to about 100% humidity, preferably at about 20% to about 99%, preferably at about 30% to about 99%, more preferably at about 40% to about 100% humidity, such as at about 50% to about 99%, such as at about 60% to about 98%, more preferably at about 70% to about 97%, more preferably at about 45% to about 80%, more preferably at about 50% to about 70%, more preferably at about 55% to about 65%, more preferably at about 92% to about 97%, or more preferably at about 94% to about 96% humidity.

In some embodiments, the disclosed cells are cultured in batch culture.

In some embodiments, the nutrient media is conditioned in a manner allowing for large scale cell growth (yielding large scale conditioned media) using, for example, an apparatus for aseptic large-scale culturing at the air-liquid interface.

Culture of Differentiated Epithelial Cells

The cells can be cultured using any number of cell/tissue culture techniques, such as using the techniques described in Rosdy M., Terminal epidermal differentiation of human keratinocytes grown in chemically defined medium on inert filter substrates at the air-liquid interface. J. Invest. Dermatology 95(4) 409-414 (October 1990). For example, cell cultures can be initiated using the 3T3 feeder-layer technique described in Green H., Kehinde O., Thomas J., Growth of cultured human epidermal cells into multiple epithelia suitable for grafting. Proc. Nat. Acad. Sci. USA 76:5665-5668 (1979) and in Rheinwald J. G., Green H.: Epidermal growth factor and the multiplication of cultured human epidermal keratinocytes. Nature 265:421-424 (1977).

In an embodiment, the inoculated substrate can be mounted, if helpful, immediately or after, e.g., 24 hours of incubation. For example, the substrate can be mounted on top of grids, such as stainless-steel grids, then cultured. The pre-conditioned medium can be deposited below the substrate in order to feed the cells only from underneath while exposing the surface of the cultures to the atmosphere. The cultures can then, for example, be incubated for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days, or any timepoint therebetween, such as from between 1 to 35 days, 2 to 34 days, 2 to 33 days, 2 to 32 days, 2 to 31 days, 2 to 30 days, 2 to 29 days, 2 to 28 days, 2 to 27 days, 2 to 26 days, 2 to 25 days, 2 to 24 days, 2 to 23 days, 2 to 22 days, 2 to 21 days, 2 to 20 days, 2 to 19 days, 2 to 18 days, 2 to 17 days, 2 to 16 days, 2 to 15 days, 2 to 14 days, 2 to 13 days, 2 to 12 days, 2 to 11 days, 2 to 10 days, 2 to 9 days, 2 to 8 days, 2 to 7 days, 3 to 16 days, 3 to 15 days, 3 to 14 days, 3 to 13 days, 3 to 12 days, 3 to 11 days, 3 to 10 days, 3 to 9 days, 3 to 8 days, 3 to 7 days, 4 to 16 days, 4 to 15 days, 4 to 14 days, 4 to 13 days, 4 to 12 days, 4 to 11 days, 4 to 10 days, 4 to 9 days, 4 to 8 days, 4 to 7 days, 5 to 16 days, 5 to 15 days, 5 to 14 days, 5 to 13 days, 5 to 12 days, 5 to 11 days, 5 to 10 days, 5 to 9 days, 5 to 8 days, or 5 to 7 days.

Differentiation in (developmental biology) refers to the normal process by which a less specialized cell undergoes maturation to become more distinct in form and function. It is also called cell differentiation. Differentiation is not limited to changes in cell size, shape, polarity, metabolism and responsiveness to signals, but can include any molecular changes that allow a progenitor cell to become more specialized and acquire a more specific role.

Thus, a “differentiated epithelial cell” as used herein refers to a keratinocyte grown in a 3-dimensional culture which is undergoing the process of differentiation over its predecessor (or progenitor) keratinocyte, wherein the predecessor keratinocyte was used to inoculate the cell cultures described herein. In an embodiment, the differentiated epithelial cell has initiated the formation of cell-to-cell connections required for one cell to grow on top of another cell to form a 3-dimensional tissue. In an embodiment, the differentiated epithelial cell and/or its descendants are capable of forming a basal layer, spinous layer, granular layer and/or corneal layer.

The term “partially differentiated epithelial cells” as used herein refers to a culture of keratinocytes grown in 3-dimensions with a thickness of living cell layers of less than about 70 μm.

The term “fully differentiated epithelial cells” as used herein refers to a culture of keratinocytes grown in 3-dimensions with a thickness of living cell layers of greater than about 70 μm and the presence of a granular layer.

The term “reconstructed epidermis” as used herein refers to a multi-layer epidermis grown in three-dimensions. In an embodiment, a reconstructed human epidermis can be formed during the third week of cell culture. For example, normal human keratinocytes can form three-dimensional reconstructed human epidermis as disclosed herein, such as the SkinEthic™ model produced by EPISKIN S.A. (www.episkin.com).

In an embodiment, the differentiated epithelial cell layers or reconstructed epidermis can comprise a clearly visible basal layer, spinous layer, granular layer and/or corneal layer.

In an embodiment, the differentiated epithelial cell layers or reconstructed epidermis has a thickness of living cell layers of at least about 2 μm, at least about 3 μm, at least about 4 μm, at least about 5 μm, at least about 6 μm, at least about 7 μm, at least about 8 μm, at least about 9 μm, at least about 10 μm, at least about 11 μm, at least about 12 μm, at least about 13 μm, at least about 14 μm, at least about 15 μm, at least about 16 μm, at least about 17 μm, at least about 18 μm, at least about 19 μm, at least about 20 μm, at least about 21 μm, at least about 22 μm, at least about 23 μm, at least about 24 μm, at least about 25 μm, at least about 26 μm, at least about 27 μm, at least about 28 μm, at least about 29 μm, at least about 30 μm, at least about 31 μm, at least about 32 μm, at least about 33 μm, at least about 34 μm, at least about 35 μm, at least about 36 μm, at least about 37 μm, at least about 38 μm, at least about 39 μm, at least about 40 μm, at least about 41 μm, at least about 42 μm, at least about 43 μm, at least about 44 μm, at least about 45 μm, at least about 46 μm, at least about 47 μm, at least about 48 μm, at least about 49 μm, at least about 50 μm, at least about 51 μm, at least about 52 μm, at least about 53 μm, at least about 54 μm, at least about 55 μm, at least about 56 μm, at least about 57 μm, at least about 58 μm, at least about 59 μm, at least about 60 μm, at least about 61 μm, at least about 62 μm, at least about 63 μm, at least about 64 μm, at least about 65 μm, at least about 66 μm, at least about 67 μm, at least about 68 μm, at least about 69 μm, at least about 70 μm, at least about 71 μm, at least about 72 μm, at least about 73 μm, at least about 74 μm, at least about 75 μm, at least about 76 μm, at least about 77 μm, at least about 78 μm, at least about 79 μm, at least about 80 μm, at least about 81 μm, at least about 82 μm, at least about 83 μm, at least about 84 μm, at least about 85 μm, at least about 86 μm, at least about 87 μm, at least about 88 μm, at least about 89 μm, at least about 90 μm, at least about 91 μm, at least about 92 μm, at least about 93 μm, at least about 94 μm, at least about 95 μm, at least about 96 μm, at least about 97 μm, at least about 98 μm, at least about 99 μm, at least about 100 μm, at least about 101 μm, at least about 102 μm, at least about 103 μm, at least about 104 μm, at least about 105 μm, at least about 106 μm, at least about 107 μm, at least about 108 μm, at least about 109 μm, at least about 110 μm, at least about 111 μm, at least about 112 μm, at least about 113 μm, at least about 114 μm, at least about 115 μm, at least about 116 μm, at least about 117 μm, at least about 118 μm, at least about 119 μm, or at least about 120 μm.

In an embodiment, the differentiated epithelial cell layers or reconstructed epidermis has a thickness of living cell layers ranging from about 2 μm to about 141 μm, from about 2 μm to about 140 μm, from about 2 μm to about 139 μm, from about 2 μm to about 138 μm, from about 2 μm to about 137 μm, from about 2 μm to about 136 μm, from about 2 μm to about 135 μm, from about 2 μm to about 134 μm, from about 2 μm to about 133 μm, from about 2 μm to about 132 μm, from about 2 μm to about 131 μm, from about 2 μm to about 130 μm, from about 2 μm to about 129 μm, from about 2 μm to about 128 μm, from about 2 μm to about 127 μm, from about 2 μm to about 126 μm, from about 2 μm to about 125 μm, from about 2 μm to about 124 μm, from about 2 μm to about 123 μm, from about 2 μm to about 122 μm, from about 2 μm to about 121 μm, or from about 2 μm to about 120 μm, and any range therebetween, such as from about 70 μm to about 141 μm, from about 70 μm to about 140 μm, from about 70 μm to about 139 μm, from about 70 μm to about 138 μm, from about 70 μm to about 137 μm, from about 70 μm to about 136 μm, from about 70 μm to about 135 μm, from about 70 μm to about 134 μm, from about 70 μm to about 133 μm, from about 70 μm to about 132 μm, from about 70 μm to about 131 μm, from about 70 μm to about 130 μm, from about 70 μm to about 129 μm, from about 70 μm to about 128 μm, from about 70 μm to about 127 μm, from about 70 μm to about 126 μm, from about 70 μm to about 125 μm, from about 70 μm to about 124 μm, from about 70 μm to about 123 μm, from about 70 μm to about 122 μm, from about 70 μm to about 121 μm, or from about 70 μm to about 120 μm, such as from about 71 μm to about 120 μm, from about 72 μm to about 120 μm, from about 73 μm to about 120 μm, from about 74 μm to about 120 μm, from about 75 μm to about 120 μm, from about 76 μm to about 120 μm, from about 77 μm to about 120 μm, from about 78 μm to about 120 μm, from about 79 μm to about 120 μm, from about 80 μm to about 120 μm, such as from about 81 μm to about 120 μm, from about 82 μm to about 120 μm, from about 83 μm to about 120 μm, from about 84 μm to about 120 μm, from about 85 μm to about 120 μm, from about 86 μm to about 120 μm, from about 87 μm to about 120 μm, from about 88 μm to about 120 μm, from about 89 μm to about 120 μm, or from about 90 μm to about 120 μm, moreover, such as from about from about 2 μm to about 70 μm, from about 2 μm to about 69 μm, from about 2 μm to about 68 μm, from about 2 μm to about 67 μm, from about 2 μm to about 66 μm, from about 2 μm to about 65 μm, from about 2 μm to about 64 μm, from about 2 μm to about 63 μm, from about 2 μm to about 62 μm, from about 2 μm to about 61 μm, from about 2 μm to about 60 μm, from about 2 μm to about 59 μm, from about 2 μm to about 58 μm, from about 2 μm to about 57 μm, from about 2 μm to about 56 μm, from about 2 μm to about 55 μm, from about 2 μm to about 54 μm, from about 2 μm to about 53 μm, from about 2 μm to about 52 μm, from about 2 μm to about 51 μm, from about 2 μm to about 50 μm, from about 2 μm to about 49 μm, from about 2 μm to about 48 μm, from about 2 μm to about 47 μm, from about 2 μm to about 46 μm, from about 2 μm to about 45 μm, from about 2 μm to about 44 μm, from about 2 μm to about 43 μm, from about 2 μm to about 42 μm, from about 2 μm to about 41 μm, from about 2 μm to about 40 μm, from about 2 μm to about 39 μm, from about 2 μm to about 38 μm, from about 2 μm to about 37 μm, from about 2 μm to about 36 μm, from about 2 μm to about 35 μm, from about 2 μm to about 34 μm, from about 2 μm to about 33 μm, from about 2 μm to about 32 μm, from about 2 μm to about 31 μm, from about 2 μm to about 30 μm, from about 2 μm to about 29 μm, from about 2 μm to about 28 μm, from about 2 μm to about 27 μm, from about 2 μm to about 26 μm, from about 2 μm to about 25 μm, from about 2 μm to about 24 μm, from about 2 μm to about 23 μm, from about 2 μm to about 22 μm, from about 2 μm to about 21 μm, from about 2 μm to about 20 μm, from about 2 μm to about 19 μm, from about 2 μm to about 18 μm, from about 2 μm to about 17 μm, from about 2 μm to about 16 μm, from about 2 μm to about 15 μm, from about 2 μm to about 14 μm, from about 2 μm to about 13 μm, from about 2 μm to about 12 μm, from about 2 μm to about 11 μm, from about 2 μm to about 10 μm, from about 2 μm to about 9 μm, from about 2 μm to about 8 μm, from about 2 μm to about 7 μm, from about 2 μm to about 6 μm, from about 2 μm to about 5 μm, from about 2 μm to about 4 μm, or from about 2 μm to about 3 μm, moreover, such as from about from about 3 μm to about 70 μm, from about 4 μm to about 70 μm, from about 5 μm to about 70 μm, from about 6 μm to about 70 μm, from about 7 μm to about 70 μm, from about 8 μm to about 70 μm, from about 9 μm to about 70 μm, from about 10 μm to about 70 μm, from about 11 μm to about 70 μm, from about 12 μm to about 70 μm, from about 13 μm to about 70 μm, from about 14 μm to about 70 μm, from about 15 μm to about 70 μm, from about 16 μm to about 70 μm, from about 17 μm to about 70 μm, from about 18 μm to about 70 μm, from about 19 μm to about 70 μm, from about 20 μm to about 70 μm, from about 21 μm to about 70 μm, from about 22 μm to about 70 μm, from about 23 μm to about 70 μm, from about 24 μm to about 70 μm, from about 25 μm to about 70 μm, from about 26 μm to about 70 μm, from about 27 μm to about 70 μm, from about 28 μm to about 70 μm, from about 29 μm to about 70 μm, from about 30 μm to about 70 μm, from about 31 μm to about 70 μm, from about 32 μm to about 70 μm, from about 33 μm to about 70 μm, from about 34 μm to about 70 μm, from about 35 μm to about 70 μm, from about 36 μm to about 70 μm, from about 37 μm to about 70 μm, from about 38 μm to about 70 μm, from about 39 μm to about 70 μm, from about 40 μm to about 70 μm, from about 41 μm to about 70 μm, from about 42 μm to about 70 μm, from about 43 μm to about 70 μm, from about 44 μm to about 70 μm, from about 45 μm to about 70 μm, from about 46 μm to about 70 μm, from about 47 μm to about 70 μm, from about 48 μm to about 70 μm, from about 49 μm to about 70 μm, from about 50 μm to about 70 μm, from about 51 μm to about 70 μm, from about 52 μm to about 70 μm, from about 53 μm to about 70 μm, from about 54 μm to about 70 μm, from about 55 μm to about 70 μm, from about 56 μm to about 70 μm, from about 57 μm to about 70 μm, from about 58 μm to about 70 μm, from about 59 μm to about 70 μm, from about 60 μm to about 70 μm, from about 61 μm to about 70 μm, from about 62 μm to about 70 μm, from about 63 μm to about 70 μm, from about 64 μm to about 70 μm, from about 65 μm to about 70 μm, from about 66 μm to about 70 μm, from about 67 μm to about 70 μm, from about 68 μm to about 70 μm, or from about 69 μm to about 70 μm.

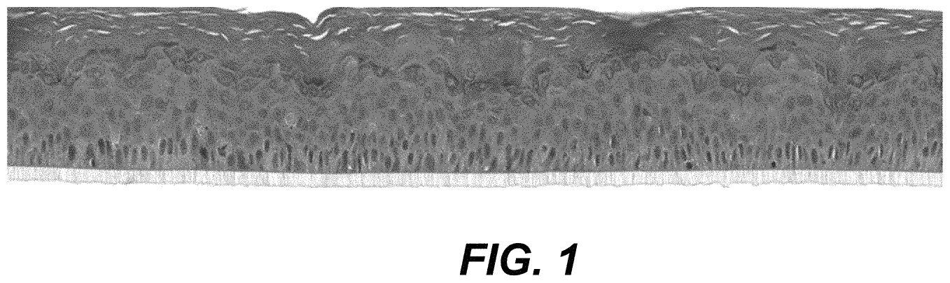

The differentiated epithelial cells approach physiologic conditions found in vivo to a greater degree than previously described with two-dimensional monolayer tissue culture systems. For example, FIG. 1 shows an embodiment wherein stained vertical sections demonstrate that the differentiated epithelial cells architecture was similar to that of normal human epidermis in vivo. In an embodiment, the characteristic differentiation pattern of epidermis can be reproduced by the differentiated epithelial cells: a stratum germinativum, a stratum spinosum, a stratum granulosum, e.g., containing many keratohyalin granules, and an anucleated stratum corneum, e.g., forming more than 10 compact cell layers. Thus, the disclosed three-dimensional cell culture supports the maturation, differentiation, and segregation of cells in culture in vitro to form components of adult epidermis analogous to counterparts found in vivo.

Collection and Processing of Conditioned Media

The term “conditioned medium” or “conditioned supernatant” as used herein refers to the cell culture medium containing extracellular protein(s) and cellular metabolites secreted by the cultured cells, which previously supported cell growth in culture. Also called “conditioned cell medium,” “conditioned cell culture medium,” “conditioned culture medium,” or “conditioned cell and tissue culture medium.” The “pre-conditioned” cell culture medium may include any defined or undefined medium that supports the growth of the desired cell type. The term “extract” when used in reference to conditioned cell culture media refers to any subcomponent or fraction of the conditioned media, whether obtained by dialysis, fractionation, distillation, phase separation, gel filtration chromatography, affinity chromatography, hollow fiber filtration, precipitation, concentration, or the like.

Conditioned media can be collected at any time during cell culture, such as at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days, such as from between 1 to 35 days, 2 to 34 days, 2 to 33 days, 2 to 32 days, 2 to 31 days, 2 to 30 days, 2 to 29 days, 2 to 28 days, 2 to 27 days, 2 to 26 days, 2 to 25 days, 2 to 24 days, 2 to 23 days, 2 to 22 days, 2 to 21 days, 2 to 20 days, 2 to 19 days, 2 to 18 days, 2 to 17 days, 2 to 16 days, 2 to 15 days, 2 to 14 days, 2 to 13 days, 2 to 12 days, 2 to 11 days, 2 to 10 days, 2 to 9 days, 2 to 8 days, 2 to 7 days, 3 to 16 days, 3 to 15 days, 3 to 14 days, 3 to 13 days, 3 to 12 days, 3 to 11 days, 3 to 10 days, 3 to 9 days, 3 to 8 days, 3 to 7 days, 4 to 16 days, 4 to 15 days, 4 to 14 days, 4 to 13 days, 4 to 12 days, 4 to 11 days, 4 to 10 days, 4 to 9 days, 4 to 8 days, 4 to 7 days, 5 to 16 days, 5 to 15 days, 5 to 14 days, 5 to 13 days, 5 to 12 days, 5 to 11 days, 5 to 10 days, 5 to 9 days, 5 to 8 days, or 5 to 7 days of cell culture. In an embodiment, the medium conditioned by the three-dimensional cell culture is collected after the cells form partially differentiated epithelial cells. In some embodiments, the epithelial cells are at least 95%, 96%, 97%, 98%, or 99% confluent when the conditioned media is collected. In some embodiments, the medium conditioned by the three-dimensional cell culture is collected after the cells form fully differentiated epithelial cells. In some embodiments, the medium conditioned by the three-dimensional cell culture is collected after the cells form at least 2 cell layers. In some embodiments, the medium conditioned by the three-dimensional cell culture is collected after the cells form a reconstructed human epithelium. In some embodiments, the medium conditioned by the three-dimensional cell culture is collected after the cells form a basal layer, a stratum spinosum, a granular layer, and a stratum corneum.

In at least some embodiments, it may be necessary to further process the conditioned medium. Once collected, the conditioned media and/or extracellular vesicles/exosomes and/or secreted biomolecules released by the 3D differentiated epithelial cells can be concentrated, frozen, freeze-dried/lyophilized, or supplemented with other additives including, but not limited to, small molecules, biologics, therapeutic agents, preservatives, or enzymes.

In some embodiments, the conditioned medium may be further processed for product isolation and purification to remove unwanted proteases, for example. The methods used for product isolation and purification so that optimal biological activity is maintained will be readily apparent to one of ordinary skill in the art. Such methods include, but are not limited to, gel chromatography (using matrices such as sephadex) ion exchange, metal chelate affinity chromatography with an insoluble matrix such as cross-linked agarose, HPLC purification and hydrophobic interaction chromatography of the conditioned media. Such techniques are described in greater detail in Cell & Tissue Culture: Laboratory Procedures, supra.

Of course, depending upon the desired application of the conditioned medium, and/or products derived thereof, appropriate measures must be taken to maintain sterility. Alternatively, sterilization may be necessary and can be accomplished by methods known to one of ordinary skill in the art, such as, for example, heat and/or filter sterilization taking care to preserve the desired biological activity.

Extracellular Vesicles

The conditioned media disclosed herein can comprise extracellular vesicles (EVs) secreted by the cultured cells. “Extracellular vesicles” or “EVs” as used herein refers to any lipid layer-delimited vesicles that are naturally released from a cell and, unlike a cell, cannot replicate. Extracellular vesicles range in diameter from near the size of the smallest physically possible unilamellar liposome (around 20-30 nanometers) to as large as 10 microns or more. EVs carry a cargo of organic molecules from the parent cell. The lysed EVs referred to herein comprise the lysed lipid layer and cargo.

A wide variety of EV subtypes have been proposed, defined variously by size, biogenesis pathway, cargo, cellular source, and function, leading to a heterogenous nomenclature. Diverse EV subtypes have been proposed, with names such as exosomes, ectosomes, microvesicles, microparticles, oncosomes, apoptotic bodies, exomeres and more. These EV subtypes have been defined by various, often overlapping, definitions, based mostly on biogenesis (cell pathway, cell or tissue identity, condition of origin). However, EV subtypes may also be defined by size, constituent molecules, function, or method of separation.

In some embodiments, extracellular vesicles can be separated from the conditioned culture so that the EV and EV cargo can be further analyzed and/or purified. A variety of separation methods can be used, including differential ultracentrifugation, density gradient ultracentrifugation, tangential flow filtration (TFF), size exclusion chromatography (SEC), ultrafiltration, capillary electrophoresis, asymmetric-flow field-flow fractionation, microfluidic methods, polymeric methods, antibody-coated microfluidic chips, and affinity/immunoaffinity capture methods. In some embodiments, tangential flow filtration and size exclusion chromatography are used. In some embodiments, the conditioned media is collected, and small extracellular vesicles are separated out using TFF. The TFF-separated small extracellular vesicles can then be concentrated and purified using SEC. SEC-purified small extracellular vesicles can then optionally be lyophilized for long-term storage at 5° C. The small extracellular vesicles can be reconstituted in a solution, such as in deionized water. In some embodiments, SEC is performed before TFF. Some embodiments do not involve a concentration step. In some embodiments, the conditioned media is collected, and large extracellular vesicles are separated out using TFF. The TFF-separated large extracellular vesicles can then be concentrated. The large extracellular vesicles can then optionally be lyophilized for long-term storage at 5° C. The large extracellular vesicles can be reconstituted in a solution, such as in deionized water. Some embodiments do not involve a concentration step. Thus, the terms “small extracellular vesicle” and “large extracellular vesicle” as used herein refer to which fraction an extracellular vesicle would be separated out in the aforementioned small EV and large EV fractions using SEC and TFF. In one embodiment, large extracellular vesicles have a diameter of more than 150 nm and small extracellular vesicles have a diameter 150 nm or less, and are separated from each other using a 200 nm filter, however large EVs and small EVs are not necessarily limited to having any particular diameter and can be present in either fraction.

In an embodiment, the ratio of the detected small extracellular vesicles to the detected large extracellular vesicles in the conditioned media is no greater than about 30.5:1, no greater than about 30:1, no greater than about 29.5:1, no greater than about 29:1, no greater than about 28.5:1, no greater than about 28:1, no greater than about 27.5:1, no greater than about 27:1, no greater than about 26.5:1, no greater than about 26:1, no greater than about 25.5:1, no greater than about 25:1, no greater than about 24.5:1, no greater than about 24:1, no greater than about 23.5:1, no greater than about 23:1, no greater than about 22.5:1, no greater than about 22:1, no greater than about 21.5:1, no greater than about 21:1, no greater than 20.5:1, such as no greater than 20:1, no greater than about 19.5:1, no greater than about 19:1, no greater than about 18.5:1, no greater than about 18:1, no greater than about 17.5:1, no greater than about 17:1, no greater than about 16.5:1, no greater than about 16:1, no greater than about 15.5:1, no greater than about 15:1, no greater than about 14.5:1, no greater than about 14:1, no greater than about 13.5:1, no greater than about 13:1, no greater than about 12.5:1, no greater than about 12:1, no greater than about 11.5:1, no greater than about 11:1, no greater than about 10.5:1, no greater than about 10:1, no greater than about 9.5:1, no greater than about 9:1, no greater than about 8.5:1, no greater than about 8:1, no greater than about 7.5:1, no greater than about 7:1, no greater than about 6.5:1, no greater than about 6:1, no greater than about 5.5:1, no greater than about 5:1, no greater than about 4.5:1, no greater than about 4:1, no greater than about 3.5:1, no greater than about 3:1, no greater than about 2.5:1, no greater than about 2:1, no greater than about 1.5:1, or no greater than about 1:1 when a) separating the small extracellular vesicles from the conditioned medium, using a combination of tangential flow filtration (TFF) and size-exclusion chromatography (SEC), then b) detecting the number of small extracellular vesicles, and a) separating the large extracellular vesicles from the conditioned medium by TFF, then b) detecting the number of large extracellular vesicles.

In further embodiments, the ratio of the detected small extracellular vesicles to the detected large extracellular vesicles in the conditioned media ranges from about 1:1 to about 30.5:1, from about 1:1 to about 30:1, from about 1:1 to about 29.5:1, from about 1:1 to about 29:1, from about 1:1 to about 28.5:1, from about 1:1 to about 28:1, from about 1:1 to about 27.5:1, from about 1:1 to about 27:1, from about 1:1 to about 26.5:1, from about 1:1 to about 26:1, from about 1:1 to about 25.5:1, from about 1:1 to about 25:1, from about 1:1 to about 24.5:1, from about 1:1 to about 24:1, from about 1:1 to about 23.5:1, from about 1:1 to about 23:1, from about 1:1 to about 22.5, from about 1:1 to about 22:1, from about 1:1 to about 21.5:1, from about 1:1 to about 21:1, from about 1:1 to about 20:1, such as from about 1:1 to about 19:1, about from 1:1 to about 18:1, from about 1:1 to about 17:1, from about 1:1 to about 16:1, from about 1:1 to about 15:1, from about 1:1 to about 14:1, from about 1:1 to about 13:1, from about 1:1 to about 12:1, from about 1:1 to about 11:1, from about 1:1 to about 10:1, from about 1:1 to about 9:1, from about 1:1 to about 8:1, from about 1:1 to about 7:1, from about 1:1 to about 6:1, from about 1:1 to about 5:1, from about 1:1 to about 4:1, from about 1:1 to about 3:1, from about 1:1 to about 2:1, from about 2:1 to about 20:1, from about 2:1 to about 19:1, from about 2:1 to about 18:1, from about 2:1 to about 17:1, from about 2:1 to about 16:1, from about 2:1 to about 15:1, from about 2:1 to about 14:1, from about 2:1 to about 13:1, from about 2:1 to about 12:1, from about 2:1 to about 11:1, from about 2:1 to about 10:1, from about 2:1 to about 9:1, from about 2:1 to about 8:1, from about 2:1 to about 7:1, from about 2:1 to about 6:1, from about 2:1 to about 5:1, from about 2:1 to about 4:1, from about 2:1 to about 3:1, from about 3:1 to about 20:1, from about 3:1 to about 19:1, from about 3:1 to about 18:1, from about 3:1 to about 17:1, from about 3:1 to about 16:1, from about 3:1 to about 15:1, from about 3:1 to about 14:1, from about 3:1 to about 13:1, from about 3:1 to about 12:1, from about 3:1 to about 11:1, from about 3:1 to about 10:1, from about 3:1 to about 9:1, from about 3:1 to about 8:1, from about 3:1 to about 7:1, from about 3:1 to about 6:1, from about 3:1 to about 5:1, from about 3:1 to about 4:1 when a) separating the small extracellular vesicles from the conditioned medium, using a combination of tangential flow filtration (TFF) and size-exclusion chromatography (SEC), then b) detecting the number of small extracellular vesicles, and a) separating the large extracellular vesicles from the conditioned medium by TFF, then b) detecting the number of large extracellular vesicles. The separated extracellular vesicles can be detected using any means including, but not limited to, nanoparticle tracking analysis (NTA) and ELISA.

Secreted Biomolecules

The conditioned medium disclosed herein can comprise a variety of secreted and excreted organic molecules/biomolecules such as carbohydrates, peptides/proteins, nucleic acids, lipids, or combinations thereof, including growth factors, metabolites, and even organelles. The term “organic molecules,” also referred to as “organic matter” or “organic materials,” as used herein refers to any molecule comprising carbon atoms bonded with other elements and/or other carbon atoms. The term “biomolecule” or “biological molecule” refers to any of numerous substances that are produced by cells and living organisms. Organic molecules and biomolecules can include carbohydrates, peptides/proteins, nucleic acids, lipids, or combinations thereof, including growth factors, metabolites, and even organelles. “Biomarker” as used herein refers to a biomolecule whose presence is indicative of some phenomenon.

The term “culture-derived” as used herein refers to a component of conditioned cell culture media that is not present in the starting cell culture media that is used to culture and feed the cells, but is produced by the cultured cells and enters the media, such as the disclosed biomolecules. Also within the meaning of the term culture-derived are compounds that are initially present in the pre-conditioned media, but whose concentration is increased during the culture process.

The disclosed keratinocyte cells and reconstructed human epithelium secrete extracellular vesicles, such as exosomes, that can comprise organic molecules/biomolecules.

Conditioned media can be tested for the presence of secreted biomolecules, or frozen at temperatures ranging from about −20° C. to −80° C. for future testing. The extracellular vesicles can also be isolated as disclosed herein, prior to assessing their biomolecule cargo.

The term “growth factor” generally refers to any protein, a polypeptide, or a complex of polypeptides, including cytokines, that are produced by a cell and which can affect the cell itself and/or a variety of other neighboring or distant cells.

In certain embodiments, the conditioned media further comprises at least one tetraspanin, such as Tetraspanin protein CD9 (CD9), Tetraspanin protein CD63 (CD63), and/or Tetraspanin protein CD81 (CD81). “Tetraspanins,” also referred to as the transmembrane 4 superfamily (TM4SF) proteins, have four transmembrane alpha-helices and two extracellular domains, one short (called the small extracellular domain or loop, SED/SEL or EC1) and one longer, typically 100 amino acid residues (the large extracellular domain/loop, LED/LEL or EC2).

The term “secretome” as used herein refers to the set of proteins expressed by an organism and secreted into the extracellular space. Subsets of the proteome may include, e.g, cytokines, growth factors, extracellular matrix proteins and regulators, and shed receptors.

The skilled artisan will be able to determine whether a particular biomolecule has been secreted by performing an appropriate assay. Exemplary assays include enzyme-linked immunosorbent assays (ELISAs), western blot, polyacrylamide gel electrophoresis, HPLC, or the like, using appropriate markers, standards, and/or commercially-available kits, as appropriate. Immunoassays using the appropriate commercially available human growth factor ELISA kits (Quantikine® Immunoassays, R & D Systems, Minneapolis, Minn.) can be performed to quantitate the concentration of various growth factors, tetraspanins and other proteins in one preparation of conditioned media. The pre-conditioned medium can be assayed in parallel as a negative (background) control.

In some embodiments, the conditioned media composition comprises a population of CD9 positive extracellular vesicles wherein the ratio of CD63 to CD81 detected in the population ranges from at least about 0.2:1, from at least about 0.3:1, from at least about 0.4:1, from at least about 0.5:1, from at least about 0.6:1, from at least about 0.7:1, from at least about 0.8:1, from at least about 0.9:1, or from at least about 1:1, or from at least about 1.1:, or from at least about 1.2:1, or from at least about 1.3:1, or from at least about 1.4:1, or from at least about 1.5:1, or from at least about 1.6:1, or from at least about 1.7:1, or from at least about 1.9:1, or from at least about 2:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

In further embodiments, the conditioned media composition comprises a population of CD9 positive extracellular vesicles wherein the ratio of CD63 to CD81 detected in the population ranges from about 0.2:1 to about 2.8:1, such as from about 0.2:1 to about 2.7:1, such as from about 0.3:1 to about 2.6:1, such as from about 0.3:1 to about 2.5:1, such as from about 0.4:1 to about 2.4:1, such as from about 0.4:1 to about 2.3:1, such as from about 0.5:1 to about 2.2:1, such as from about 0.6:1 to about 2.1:1, such as from about 0.6:1 to about 2.0:1, such as from about 0.6:1 to about 1.9:1, such as from about 0.7:1 to about 1.8:1, such as from about 0.7:1 to about 1.7:1, such as from about 0.8:1 to about 1.6:1, such as from about 0.8:1 to about 1.5:1, from about 0.9:1 to about 1.4:1, from about 0.9:1 to about 1.3:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

Cosmetic and Medical Compositions

In some embodiments, the conditioned cell media and/or its secretions can be used in the preparation of medical and cosmetic applications.

In certain applications, for example, the compositions may affect the underlying structure of the skin, decrease wrinkle depth, and/or reverse or ameliorate the effect of photooxidation or aging on the skin.

The compositions may, in various embodiments, be particularly useful as skin care products, hair care products, and sun care products.

The compositions disclosed herein may also be “medical compositions” that provide medicinal or drug-like benefits.

In certain embodiments, compositions comprise delivery systems including at least one of liposomes, cyclodextrins, polymer systems, or hyaluronic acid or related compounds. The compositions comprise cosmetically-acceptable carriers. The skilled artisan will understand that a pharmaceutically-acceptable carrier or formulation that is suitable for topical applications will typically also be a cosmetically-acceptable carrier or formulation.

A topical cosmetic or medical ointment, lotion, or gel composition typically contains a concentration of active ingredients comprising conditioned media or extracts thereof, from about 1 to 99%, about 5 to 95%, about 20 to 75%, or about 5 to 20%, in a cosmetically-acceptable carrier (which may also be a medically-acceptable carrier), such as a pharmaceutical cream base, an oil-in-water emulsion, a water-in-oil emulsion, a gel, or the like. Various compositions for topical use include drops, tinctures, lotions, creams, salves, serums, solutions, and ointments containing conditioned media or extracts, and an appropriate carrier.

In certain embodiments, the disclosed compositions are in the form of lotions, creams, gels, including hydrogels, powders, serums, salves, foundations, facial masks, lip care products, sunscreens, hair care products, skin cleansers, exfoliants, compact formulations, or the like. The optimal percentage of the conditioned media or extract in each composition varies according to the composition's formulation and the therapeutic effect desired.

The skilled artisan will understand that the appropriate carriers of the inventive compositions typically will contain ingredients, such as those typically found in the cosmetic fields: oils, waxes or other standard fatty substances, or conventional gelling agents and/or thickeners; emulsifiers; moisturizing agents; emollients; sunscreens; hydrophilic or lipophilic active agents, such as ceramides; agents for combatting free radicals; bactericides; sequestering agents; preservatives; basifying or acidifying agents; fragrances; surfactants; fillers; natural products or extracts of natural product, such as aloe or green tea extract; vitamins; or coloring materials. The amounts of these various ingredients will vary depending on the use of the composition and the cosmetic or medical effect desired.

Discussions of cosmetically-acceptable ingredients and formulations may be found in, among other places, FDA Cosmetics Handbook, U.S. Food and Drug Administration; Handbook of Cosmetic and Personal Care Additives, Ash and Ash, compilers, 1994, Chemical Publishing, New York, N.Y.; Bennett's Cosmetic Formulary, 1993, Chemical Publishing Co.; Harry's Cosmeticology, 7th ed., Wilkinson & Moore, 1982 and 8th ed., Rieger, 2000, Chemical Publishing; Cosmetic Bench Reference-2001, Allerud Publishing Corp.; CTFA Compendium of Cosmetic Ingredient Composition, Nikitakis and McEwen, eds., 1990, Cosmetic, Toiletry, and Fragrance Association, Washington, D.C., Surfactant Encyclopedia, 2nd revised edition, Rieger, 1996, Allured Publishing; The Chemistry and Manufacture of Cosmetics, 2nd ed., De Navarre, Van Nostrand, Princeton, N.J.; Encyclopedia of Common Natural Ingredients Used in Food, Drugs, and Cosmetics, Leung, 1996, John Wiley; A Consumer's Dictionary of Cosmetic Ingredients, 5th ed., Winter, 1999, Three Rivers Press, New York, N.Y.; Cosmeceuticals: Active Skin Treatment, 1998, Allured Publishing; Handbook of Cosmetic Science and Technology, Knowlton and Pearce, 1993, Elsevier Advanced Technology, Oxford, UK; Personal-Care Formulas, 1997, Allured Publishing; Beginning Cosmetic Chemistry, Scheuller and Romanowski, 1999, Allured Publishing; and Skin Permeation: Fundamentals and Application, Zatz, 1993, Allured Publishing. Discussions of pharmaceutically-acceptable ingredients and formulations may be found in, among other places, Remington's Pharmaceutical Sciences, 18th ed., Gennaro, ed., 1990, Mack Publishing.

“Cosmetically treated” (and its grammatical variations) means treated to improve or restore appearance.

“Medically treated” (and its grammatical variations) as used herein refers to the improvement of at least one biomarker for good health.

“Topical application” (and its grammatical variations) as used herein refers to the application of the compositions of the present disclosure onto keratinous substrates such as skin.

Disclosed herein are methods of treatment comprising applying the disclosed compositions to epidermal cells or skin. Methods of growing epidermal cells comprising applying the disclosed compositions to an epidermis are also disclosed. In some embodiments, methods for promoting fibroblast growth comprising applying the disclosed compositions to skin or epidermal cells are also disclosed. In some embodiments, methods for increasing keratinocyte growth factor secretion or release comprising applying a composition according to the disclosure to skin are also disclosed. In some embodiments, methods for inducing keratinocyte growth factor secretion or release comprising applying a composition according to the disclosure to skin are also disclosed.

### Exemplary Embodiments

The following exemplary and non-limiting embodiments of the disclosed subject matter are contemplated.

According to a first (1) embodiment, the disclosed composition may comprise extracellular vesicles and an acceptable cosmetic carrier, wherein the extracellular vesicles are derived from a conditioned medium collected from differentiated epithelial cells, predecessor keratinocytes, or combinations thereof cultured at an air-liquid interface in a nutrient medium sufficient to meet the nutritional needs required to grow the cells in vitro to form differentiated epithelial cells.

According to a second (2) embodiment, in an embodiment of embodiments 1 or 80, the differentiated epithelial cells are at least 95%, 96%, 97%, 98%, or 99% confluent.

According to a third (3) embodiment, in an embodiment of any one of embodiments 1, 2 or 80, the differentiated epithelial cells may comprise on average at least 2, 3, 4, 5, 6, 7, 8, 9, 10, 11, 12, 13, 14, 15, 16, 17, 18, 19, 20, 21, 22, 23, 24, or 25 living cell layers.

According to a fourth (4) embodiment, in an embodiment of any one of embodiments 1-3 or 80, the differentiated epithelial cells may comprise on average 1 to 2 cell layers, such as 2 living cell layers.

According to a fifth (5) embodiment, in an embodiment of any one of embodiments 1-3 or 80, the differentiated epithelial cells may comprise on average 3 to 5 cell layers, such as 3 to 4 living cell layers.

According to a sixth (6) embodiment, in an embodiment of any one of embodiments 1-3 or 80, the differentiated epithelial cells may comprise on average 5 to 8 cell layers, such as 6 to 7 living cell layers.

According to a seventh (7) embodiment, in an embodiment of any one of embodiments 1-3 or 80, the differentiated epithelial cells may comprise on average 1 to 8 living cell layers.

According to an eighth (8) embodiment, in an embodiment of any one of embodiments 1-3, 6-7 or 80, the differentiated epithelial cells may form cell layers comprising a basal layer, a stratum spinosum, a granular layer, and/or a stratum corneum.

According to a ninth (9) embodiment, in an embodiment of any one of embodiments 1-8 or 80, the differentiated epithelial cells may have a thickness of living cell layers of at least about 2 μm, at least about 3 μm, at least about 4 μm, at least about 5 μm, at least about 6 μm, at least about 7 μm, at least about 8 μm, at least about 9 μm, at least about 10 μm, at least about 11 μm, at least about 12 μm, at least about 13 μm, at least about 14 μm, at least about 15 μm, at least about 16 μm, at least about 17 μm, at least about 18 μm, at least about 19 μm, at least about 20 μm, at least about 21 μm, at least about 22 μm, at least about 23 μm, at least about 24 μm, at least about 25 μm, at least about 26 μm, at least about 27 μm, at least about 28 μm, at least about 29 μm, at least about 30 μm, at least about 31 μm, at least about 32 μm, at least about 33 μm, at least about 34 μm, at least about 35 μm, at least about 36 μm, at least about 37 μm, at least about 38 μm, at least about 39 μm, at least about 40 μm, at least about 41 μm, at least about 42 μm, at least about 43 μm, at least about 44 μm, at least about 45 μm, at least about 46 μm, at least about 47 μm, at least about 48 μm, at least about 49 μm, at least about 50 μm, at least about 51 μm, at least about 52 μm, at least about 53 μm, at least about 54 μm, at least about 55 μm, at least about 56 μm, at least about 57 μm, at least about 58 μm, at least about 59 μm, at least about 60 μm, at least about 61 μm, at least about 62 μm, at least about 63 μm, at least about 64 μm, at least about 65 μm, at least about 66 μm, at least about 67 μm, at least about 68 μm, at least about 69 μm, at least about 70 μm, at least about 71 μm, at least about 72 μm, at least about 73 μm, at least about 74 μm, at least about 75 μm, at least about 76 μm, at least about 77 μm, at least about 78 μm, at least about 79 μm, at least about 80 μm, at least about 81 μm, at least about 82 μm, at least about 83 μm, at least about 84 μm, at least about 85 μm, at least about 86 μm, at least about 87 μm, at least about 88 μm, at least about 89 μm, at least about 90 μm, at least about 91 μm, at least about 92 μm, at least about 93 μm, at least about 94 μm, at least about 95 μm, at least about 96 μm, at least about 97 μm, at least about 98 μm, at least about 99 μm, at least about 100 μm, at least about 101 μm, at least about 102 μm, at least about 103 μm, at least about 104 μm, at least about 105 μm, at least about 106 μm, at least about 107 μm, at least about 108 μm, at least about 109 μm, at least about 110 μm, at least about 111 μm, at least about 112 μm, at least about 113 μm, at least about 114 μm, at least about 115 μm, at least about 116 μm, at least about 117 μm, at least about 118 μm, at least about 119 μm, or at least about 120 μm.

According to a tenth (10) embodiment, in an embodiment of any one of embodiments 1-3 or 80, the differentiated epithelial cells may have a thickness of living cell layers of about 2 μm to about 141 μm, from about 2 μm to about 140 μm, from about 2 μm to about 139 μm, from about 2 μm to about 138 μm, from about 2 μm to about 137 μm, from about 2 μm to about 136 μm, from about 2 μm to about 135 μm, from about 2 μm to about 134 μm, from about 2 μm to about 133 μm, from about 2 μm to about 132 μm, from about 2 μm to about 131 μm, from about 2 μm to about 130 μm, from about 2 μm to about 129 μm, from about 2 μm to about 128 μm, from about 2 μm to about 127 μm, from about 2 μm to about 126 μm, from about 2 μm to about 125 μm, from about 2 μm to about 124 μm, from about 2 μm to about 123 μm, from about 2 μm to about 122 μm, from about 2 μm to about 121 μm, or from about 2 μm to about 120 μm.

According to an 11^{th} (11) embodiment, in an embodiment of any one of embodiments 1-3 or 80, the differentiated epithelial cells may have a thickness of living cell layers of from about 70 μm to about 141 μm, from about 70 μm to about 140 μm, from about 70 μm to about 139 μm, from about 70 μm to about 138 μm, from about 70 μm to about 137 μm, from about 70 μm to about 136 μm, from about 70 μm to about 135 μm, from about 70 μm to about 134 μm, from about 70 μm to about 133 μm, from about 70 μm to about 132 μm, from about 70 μm to about 131 μm, from about 70 μm to about 130 μm, from about 70 μm to about 129 μm, from about 70 μm to about 128 μm, from about 70 μm to about 127 μm, from about 70 μm to about 126 μm, from about 70 μm to about 125 μm, from about 70 μm to about 124 μm, from about 70 μm to about 123 μm, from about 70 μm to about 122 μm, from about 70 μm to about 121 μm, or from about 70 μm to about 120 μm, such as from about 71 μm to about 120 μm, from about 72 μm to about 120 μm, from about 73 μm to about 120 μm, from about 74 μm to about 120 μm, from about 75 μm to about 120 μm, from about 76 μm to about 120 μm, from about 77 μm to about 120 μm, from about 78 μm to about 120 μm, from about 79 μm to about 120 μm, from about 80 μm to about 120 μm, such as from about 81 μm to about 120 μm, from about 82 μm to about 120 μm, from about 83 μm to about 120 μm, from about 84 μm to about 120 μm, from about 85 μm to about 120 μm, from about 86 μm to about 120 μm, from about 87 μm to about 120 μm, from about 88 μm to about 120 μm, from about 89 μm to about 120 μm, or from about 90 μm to about 120 μm.

According to a 12^{th} (12) embodiment, in an embodiment of any one of embodiments 1-3 or 80, the differentiated epithelial cells have a thickness of living cell layers of from about 2 μm to about 70 μm, from about 2 μm to about 69 μm, from about 2 μm to about 68 μm, from about 2 μm to about 67 μm, from about 2 μm to about 66 μm, from about 2 μm to about 65 μm, from about 2 μm to about 64 μm, from about 2 μm to about 63 μm, from about 2 μm to about 62 μm, from about 2 μm to about 61 μm, from about 2 μm to about 60 μm, from about 2 μm to about 59 μm, from about 2 μm to about 58 μm, from about 2 μm to about 57 μm, from about 2 μm to about 56 μm, from about 2 μm to about 55 μm, from about 2 μm to about 54 μm, from about 2 μm to about 53 μm, from about 2 μm to about 52 μm, from about 2 μm to about 51 μm, from about 2 μm to about 50 μm, from about 2 μm to about 49 μm, from about 2 μm to about 48 μm, from about 2 μm to about 47 μm, from about 2 μm to about 46 μm, from about 2 μm to about 45 μm, from about 2 μm to about 44 μm, from about 2 μm to about 43 μm, from about 2 μm to about 42 μm, from about 2 μm to about 41 μm, from about 2 μm to about 40 μm, from about 2 μm to about 39 μm, from about 2 μm to about 38 μm, from about 2 μm to about 37 μm, from about 2 μm to about 36 μm, from about 2 μm to about 35 μm, from about 2 μm to about 34 μm, from about 2 μm to about 33 μm, from about 2 μm to about 32 μm, from about 2 μm to about 31 μm, from about 2 μm to about 30 μm, from about 2 μm to about 29 μm, from about 2 μm to about 28 μm, from about 2 μm to about 27 μm, from about 2 μm to about 26 μm, from about 2 μm to about 25 μm, from about 2 μm to about 24 μm, from about 2 μm to about 23 μm, from about 2 μm to about 22 μm, from about 2 μm to about 21 μm, from about 2 μm to about 20 μm, from about 2 μm to about 19 μm, from about 2 μm to about 18 μm, from about 2 μm to about 17 μm, from about 2 μm to about 16 μm, from about 2 μm to about 15 μm, from about 2 μm to about 14 μm, from about 2 μm to about 13 μm, from about 2 μm to about 12 μm, from about 2 μm to about 11 μm, from about 2 μm to about 10 μm, from about 2 μm to about 9 μm, from about 2 μm to about 8 μm, from about 2 μm to about 7 μm, from about 2 μm to about 6 μm, from about 2 μm to about 5 μm, from about 2 μm to about 4 μm, or from about 2 μm to about 3 μm.

According to a 13^{th} (13) embodiment, in an embodiment of any one of embodiments 1-3 or 80, the differentiated epithelial cells may have a thickness of living cell layers of from about 3 μm to about 70 μm, from about 4 μm to about 70 μm, from about 5 μm to about 70 μm, from about 6 μm to about 70 μm, from about 7 μm to about 70 μm, from about 8 μm to about 70 μm, from about 9 μm to about 70 μm, from about 10 μm to about 70 μm, from about 11 μm to about 70 μm, from about 12 μm to about 70 μm, from about 13 μm to about 70 μm, from about 14 μm to about 70 μm, from about 15 μm to about 70 μm, from about 16 μm to about 70 μm, from about 17 μm to about 70 μm, from about 18 μm to about 70 μm, from about 19 μm to about 70 μm, from about 20 μm to about 70 μm, from about 21 μm to about 70 μm, from about 22 μm to about 70 μm, from about 23 μm to about 70 μm, from about 24 μm to about 70 μm, from about 25 μm to about 70 μm, from about 26 μm to about 70 μm, from about 27 μm to about 70 μm, from about 28 μm to about 70 μm, from about 29 μm to about 70 μm, from about 30 μm to about 70 μm, from about 31 μm to about 70 μm, from about 32 μm to about 70 μm, from about 33 μm to about 70 μm, from about 34 μm to about 70 μm, from about 35 μm to about 70 μm, from about 36 μm to about 70 μm, from about 37 μm to about 70 μm, from about 38 μm to about 70 μm, from about 39 μm to about 70 μm, from about 40 μm to about 70 μm, from about 41 μm to about 70 μm, from about 42 μm to about 70 μm, from about 43 μm to about 70 μm, from about 44 μm to about 70 μm, from about 45 μm to about 70 μm, from about 46 μm to about 70 μm, from about 47 μm to about 70 μm, from about 48 μm to about 70 μm, from about 49 μm to about 70 μm, from about 50 μm to about 70 μm, from about 51 μm to about 70 μm, from about 52 μm to about 70 μm, from about 53 μm to about 70 μm, from about 54 μm to about 70 μm, from about 55 μm to about 70 μm, from about 56 μm to about 70 μm, from about 57 μm to about 70 μm, from about 58 μm to about 70 μm, from about 59 μm to about 70 μm, from about 60 μm to about 70 μm, from about 61 μm to about 70 μm, from about 62 μm to about 70 μm, from about 63 μm to about 70 μm, from about 64 μm to about 70 μm, from about 65 μm to about 70 μm, from about 66 μm to about 70 μm, from about 67 μm to about 70 μm, from about 68 μm to about 70 μm, or from about 69 μm to about 70 μm.

According to a 14^{th} (14) embodiment, in an embodiment of any one of embodiments 1-13 or 80, the differentiated epithelial cells have differentiated over predecessor mammalian keratinocytes.

According to a 15^{th} (15) embodiment, in an embodiment of any one of embodiments 1-14 or 80, the differentiated epithelial cells have differentiated over predecessor human keratinocytes.

According to a 16^{th} (16) embodiment, in an embodiment of any one of embodiments 1-15 or 80, the differentiated epithelial cells may be cultured for at least 1, 2, 3, 4, 5, 6, 7, 8, 9, 10, 11, 12, 13 14, 15, 16, 17, 18, 19, 20, 21, 22, 23, 24, 25, 26, 27, 28, 29, 30, 31, 32, 33, 34, or 35 days, such as from between 1 to 35 days, 2 to 34 days, 2 to 33 days, 2 to 32 days, 2 to 31 days, 2 to 30 days, 2 to 29 days, 2 to 28 days, 2 to 27 days, 2 to 26 days, 2 to 25 days, 2 to 24 days, 2 to 23 days, 2 to 22 days, 2 to 21 days, 2 to 20 days, 2 to 19 days, 2 to 18 days, 2 to 17 days, 2 to 16 days, 2 to 15 days, 2 to 14 days, 2 to 13 days, 2 to 12 days, 2 to 11 days, 2 to 10 days, 2 to 9 days, 2 to 8 days, 2 to 7 days, 3 to 16 days, 3 to 15 days, 3 to 14 days, 3 to 13 days, 3 to 12 days, 3 to 11 days, 3 to 10 days, 3 to 9 days, 3 to 8 days, 3 to 7 days, 4 to 16 days, 4 to 15 days, 4 to 14 days, 4 to 13 days, 4 to 12 days, 4 to 11 days, 4 to 10 days, 4 to 9 days, 4 to 8 days, 4 to 7 days, 5 to 16 days, 5 to 15 days, 5 to 14 days, 5 to 13 days, 5 to 12 days, 5 to 11 days, 5 to 10 days, 5 to 9 days, 5 to 8 days, or 5 to 7 days.

According to a 17^{th} (17) embodiment, in an embodiment of any one of embodiments 1-16 or 80, the differentiated epithelial cells may be cultured on a porous substrate.

According to an 18^{th} (18) embodiment, in an embodiment of any one of embodiments 1-17 or 80, wherein the differentiated epithelial cells may be incubated for at least 3 hours.

According to a 19^{th} (19) embodiment, in an embodiment of any one of embodiments 1-18 or 80, the differentiated epithelial cells may be incubated at a temperature ranging from about 36° C. to about 38° C.

According to a 20^{th} (20) embodiment, in an embodiment of any one of embodiments 1-19 or 80, the differentiated epithelial cells may be incubated at about 4%-6% CO2.

According to a 21^{st} (21) embodiment, in an embodiment of any one of embodiments 1-20 or 80, the differentiated epithelial cells may be incubated at about 40% to about 100% humidity, preferably at about 50% to about 99%, preferably at about 60% to about 99%, preferably at about 70% to about 99%, preferably at about 80% to about 99%, preferably at about 85% to about 99%, at about 90% to about 98%, at about 92% to about 97%, or at about 94% to about 96% humidity.

According to a 22^{nd} (22) embodiment, in an embodiment of any one of embodiments 1-21 or 80, the conditioned medium comprising the extracellular vesicles may be concentrated, filtered, and/or purified prior to combining the extracellular vesicles with an acceptable carrier to form the composition.

According to a 23^{rd} (23) embodiment, in an embodiment of any one of embodiments 1-22 or 80, the extracellular vesicles may be isolated from the conditioned media prior to combining the extracellular vesicles with an acceptable carrier to form the composition.

According to a 24^{th} (24) embodiment, in an embodiment of any one of embodiments 1-23 or 80, the extracellular vesicles may be lysed prior to combination with an acceptable carrier.

According to a 25^{th} (25) embodiment, in an embodiment of any one of embodiments 1-24 or 80, the nutrient medium may be a chemically defined medium.

According to a 26^{th} (26) embodiment, in an embodiment of any one of embodiments 1-25 or 80, the differentiated epithelial cells may be cultured in batch culture.

According to a 27^{th} (27) embodiment, in an embodiment of any one of embodiments 1-26 or 80, the extracellular vesicles may comprise large extracellular vesicles having a diameter of more than 150 nm and small extracellular vesicles having a diameter 150 nm or less, wherein the ratio of the small extracellular vesicles to the large extracellular vesicles is no greater than about 30.5:1, no greater than about 30:1, no greater than about 29.5:1, no greater than about 29:1, no greater than about 28.5:1, no greater than about 28:1, no greater than about 27.5:1, no greater than about 27:1, no greater than about 26.5:1, no greater than about 26:1, no greater than about 25.5:1, no greater than about 25:1, no greater than about 24.5:1, no greater than about 24:1, no greater than about 23.5:1, no greater than about 23:1, no greater than about 22.5:1, no greater than about 22:1, no greater than about 21.5:1 no greater than about 21:1, no greater than about 20.5:1, no greater than about 20:1, no greater than about 19.5:1, no greater than about 19:1, no greater than about 18.5:1, no greater than about 18:1, no greater than about 17.5:1, no greater than about 17:1, no greater than about 16.5:1, no greater than about 16:1, no greater than about 15.5:1, no greater than about 15:1, no greater than about 14.5:1, no greater than about 14:1, no greater than about 13.5:1, no greater than about 13:1, no greater than about 12.5:1, no greater than about 12:1, no greater than about 11.5:1, no greater than about 11:1, no greater than about 10.5:1, no greater than about 10:1, no greater than about 9.5:1, no greater than about 9:1, no greater than about 8.5:1, no greater than about 8:1, no greater than about 7.5:1, no greater than about 7:1, no greater than about 6.5:1, no greater than about 6:1, no greater than about 5.5:1, no greater than about 5:1, no greater than about 4.5:1, no greater than about 4:1, no greater than about 3.5:1, no greater than about 3:1, no greater than about 2.5:1, no greater than about 2:1, no greater than about 1.5:1, or no greater than about 1:1 when a) separating the small extracellular vesicles from the conditioned medium, using a combination of tangential flow filtration (TFF) and size-exclusion chromatography (SEC), then b) detecting the number of small extracellular vesicles, and a) separating the large extracellular vesicles from the conditioned medium by TFF, then b) detecting the number of large extracellular vesicles.

According to a 28^{th} (28) embodiment, in an embodiment of any one of embodiments 1-27 or 80, the extracellular vesicles may comprise large extracellular vesicles having a diameter of more than 150 nm and small extracellular vesicles having a diameter 150 nm or less, wherein the average ratio of the small extracellular vesicles to the large extracellular vesicles ranges from about 1:1 to about 30.5:1, from about 1:1 to about 30:1, from about 1:1 to about 29.5:1, from about 1:1 to about 29:1, from about 1:1 to about 28.5:1, from about 1:1 to about 28:1, from about 1:1 to about 27.5:1, from about 1:1 to about 27:1, from about 1:1 to about 26.5:1, from about 1:1 to about 26:1, from about 1:1 to about 25.5:1, from about 1:1 to about 25:1, from about 1:1 to about 24.5:1, from about 1:1 to about 24:1, from about 1:1 to about 23.5:1, from about 1:1 to about 23:1, from about 1:1 to about 22.5, from about 1:1 to about 22:1, from about 1:1 to about 21.5:1, from about 1:1 to about 21:1, from about 1:1 to about 20:1, from about 1:1 to about 19:1, about from 1:1 to about 18:1, from about 1:1 to about 17:1, from about 1:1 to about 16:1, from about 1:1 to about 15:1, from about 1:1 to about 14:1, from about 1:1 to about 13:1, from about 1:1 to about 12:1, from about 1:1 to about 11:1, from about 1:1 to about 10:1, from about 1:1 to about 9:1, from about 1:1 to about 8:1, from about 1:1 to about 7:1, from about 1:1 to about 6:1, from about 1:1 to about 5:1, from about 1:1 to about 4:1, from about 1:1 to about 3:1, from about 1:1 to about 2:1, from about 2:1 to about 20:1, from about 2:1 to about 19:1, from about 2:1 to about 18:1, from about 2:1 to about 17:1, from about 2:1 to about 16:1, from about 2:1 to about 15:1, from about 2:1 to about 14:1, from about 2:1 to about 13:1, from about 2:1 to about 12:1, from about 2:1 to about 11:1, from about 2:1 to about 10:1, from about 2:1 to about 9:1, from about 2:1 to about 8:1, from about 2:1 to about 7:1, from about 2:1 to about 6:1, from about 2:1 to about 5:1, from about 2:1 to about 4:1, from about 2:1 to about 3:1, from about 3:1 to about 20:1, from about 3:1 to about 19:1, from about 3:1 to about 18:1, from about 3:1 to about 17:1, from about 3:1 to about 16:1, from about 3:1 to about 15:1, from about 3:1 to about 14:1, from about 3:1 to about 13:1, from about 3:1 to about 12:1, from about 3:1 to about 11:1, from about 3:1 to about 10:1, from about 3:1 to about 9:1, from about 3:1 to about 8:1, from about 3:1 to about 7:1, from about 3:1 to about 6:1, from about 3:1 to about 5:1, or from about 3:1 to about 4:1 when a) separating the small extracellular vesicles from the conditioned medium, using a combination of tangential flow filtration (TFF) and size-exclusion chromatography (SEC), then b) detecting the number of small extracellular vesicles, and a) separating the large extracellular vesicles from the conditioned medium by TFF, then b) detecting the number of large extracellular vesicles.

According to a 29^{th} (29) embodiment, in an embodiment of any one of embodiments 1-28 or 80, the composition may comprise a population of CD9 positive extracellular vesicles wherein the ratio of CD63 to CD81 detected in the population is from at least about 0.2:1, from at least about 0.3:1, from at least about 0.4:1, from at least about 0.5:1, from at least about 0.6:1, from at least about 0.7:1, from at least about 0.8:1, from at least about 0.9:1, or from at least about 1:1, or from at least about 1.1:, or from at least about 1.2:1, or from at least about 1.3:1, or from at least about 1.4:1, or from at least about 1.5:1, or from at least about 1.6:1, or from at least about 1.7:1, or from at least about 1.9:1, or from at least about 2:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

According to a 30^{th} (30) embodiment, in an embodiment of any one of embodiments 1-29 or 80, the composition may comprise a population of CD9 positive extracellular vesicles wherein the ratio of CD63 to CD81 detected in the population ranges from about 0.2:1 to about 2.8:1, from about 0.2:1 to about 2.7:1, from about 0.3:1 to about 2.6:1, from about 0.3:1 to about 2.5:1, from about 0.4:1 to about 2.4:1, from about 0.4:1 to about 2.3:1, from about 0.5:1 to about 2.2:1, from about 0.6:1 to about 2.1:1, from about 0.6:1 to about 2.0:1, from about 0.6:1 to about 1.9:1, from about 0.7:1 to about 1.8:1, from about 0.7:1 to about 1.7:1, from about 0.8:1 to about 1.6:1, from about 0.8:1 to about 1.5:1, from about 0.9:1 to about 1.4:1, or from about 0.9:1 to about 1.3:1 when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

According to a 31^{st} (31) embodiment, in an embodiment of any one of embodiments 1-30 or 80, the composition may further comprise small molecules, biologics, therapeutic agents, preservatives, or enzymes.

A 32^{nd} (32) embodiment comprises the disclosed method of making a composition comprising combining extracellular vesicles with an acceptable carrier to form the composition, wherein the extracellular vesicles are derived from a conditioned medium collected from differentiated epithelial cells, predecessor keratinocytes, or combinations thereof cultured at an air-liquid interface in a nutrient medium sufficient to meet the nutritional needs required to grow the cells in vitro to form differentiated epithelial cells.

According to a 33^{rd} (33) embodiment, in an embodiment of embodiment 32, the differentiated epithelial cells may be at least 95%, 96%, 97%, 98%, or 99% confluent.

According to a 34^{th} (34) embodiment, in an embodiment of embodiments 32 or 33, the differentiated epithelial cells may comprise on average at least 2, 3, 4, 5, 6, 7, 8, 9, 10, 11, 12, 13, 14, 15, 16, 17, 18, 19, 20, 21, 22, 23, 24, or 25 living cell layers.

According to a 35^{th} (35) embodiment, in an embodiment of any one of embodiments 32-34, the differentiated epithelial cells may comprise on average 1 to 2 cell layers, such as 2 living cell layers.

According to a 36^{th} (36) embodiment, in an embodiment of any one of embodiments 32-34, the differentiated epithelial cells may comprise on average 3 to 5 cell layers, such as 3 to 4 living cell layers.

According to a 37^{th} (37) embodiment, in an embodiment of any one of embodiments 32-34, the differentiated epithelial cells may comprise on average 5 to 8 cell layers, such as 6 to 7 living cell layers.

According to a 38^{th} (38) embodiment, in an embodiment of any one of embodiments 32-34, the differentiated epithelial cells may comprise on average 1 to 8 living cell layers.

According to a 39^{th} (39) embodiment, in an embodiment of any one of embodiments 32-34 or 37-38, the differentiated epithelial cells may form cell layers comprising a basal layer, a stratum spinosum, a granular layer, and/or a stratum corneum.

According to a 40^{th} (40) embodiment, in an embodiment of any one of embodiments 32-38, the differentiated epithelial cells may have a thickness of living cell layers of at least about 2 μm, at least about 3 μm, at least about 4 μm, at least about 5 μm, at least about 6 μm, at least about 7 μm, at least about 8 μm, at least about 9 μm, at least about 10 μm, at least about 11 μm, at least about 12 μm, at least about 13 μm, at least about 14 μm, at least about 15 μm, at least about 16 μm, at least about 17 μm, at least about 18 μm, at least about 19 μm, at least about 20 μm, at least about 21 μm, at least about 22 μm, at least about 23 μm, at least about 24 μm, at least about 25 μm, at least about 26 μm, at least about 27 μm, at least about 28 μm, at least about 29 μm, at least about 30 μm, at least about 31 μm, at least about 32 μm, at least about 33 μm, at least about 34 μm, at least about 35 μm, at least about 36 μm, at least about 37 μm, at least about 38 μm, at least about 39 μm, at least about 40 μm, at least about 41 μm, at least about 42 μm, at least about 43 μm, at least about 44 μm, at least about 45 μm, at least about 46 μm, at least about 47 μm, at least about 48 μm, at least about 49 μm, at least about 50 μm, at least about 51 μm, at least about 52 μm, at least about 53 μm, at least about 54 μm, at least about 55 μm, at least about 56 μm, at least about 57 μm, at least about 58 μm, at least about 59 μm, at least about 60 μm, at least about 61 μm, at least about 62 μm, at least about 63 μm, at least about 64 μm, at least about 65 μm, at least about 66 μm, at least about 67 μm, at least about 68 μm, at least about 69 μm, at least about 70 μm, at least about 71 μm, at least about 72 μm, at least about 73 μm, at least about 74 μm, at least about 75 μm, at least about 76 μm, at least about 77 μm, at least about 78 μm, at least about 79 μm, at least about 80 μm, at least about 81 μm, at least about 82 μm, at least about 83 μm, at least about 84 μm, at least about 85 μm, at least about 86 μm, at least about 87 μm, at least about 88 μm, at least about 89 μm, at least about 90 μm, at least about 91 μm, at least about 92 μm, at least about 93 μm, at least about 94 μm, at least about 95 μm, at least about 96 μm, at least about 97 μm, at least about 98 μm, at least about 99 μm, at least about 100 μm, at least about 101 μm, at least about 102 μm, at least about 103 μm, at least about 104 μm, at least about 105 μm, at least about 106 μm, at least about 107 μm, at least about 108 μm, at least about 109 μm, at least about 110 μm, at least about 111 μm, at least about 112 μm, at least about 113 μm, at least about 114 μm, at least about 115 μm, at least about 116 μm, at least about 117 μm, at least about 118 μm, at least about 119 μm, or at least about 120 μm.

According to a 41^{st} (41) embodiment, in an embodiment of any one of embodiments 32-34, the differentiated epithelial cells may have a thickness of living cell layers of about 2 μm to about 141 μm, from about 2 μm to about 140 μm, from about 2 μm to about 139 μm, from about 2 μm to about 138 μm, from about 2 μm to about 137 μm, from about 2 μm to about 136 μm, from about 2 μm to about 135 μm, from about 2 μm to about 134 μm, from about 2 μm to about 133 μm, from about 2 μm to about 132 μm, from about 2 μm to about 131 μm, from about 2 μm to about 130 μm, from about 2 μm to about 129 μm, from about 2 μm to about 128 μm, from about 2 μm to about 127 μm, from about 2 μm to about 126 μm, from about 2 μm to about 125 μm, from about 2 μm to about 124 μm, from about 2 μm to about 123 μm, from about 2 μm to about 122 μm, from about 2 μm to about 121 μm, or from about 2 μm to about 120 μm.

According to a 42^{nd} (42) embodiment, in an embodiment of any one of embodiments 32-34, the differentiated epithelial cells may have a thickness of living cell layers of from about 70 μm to about 141 μm, from about 70 μm to about 140 μm, from about 70 μm to about 139 μm, from about 70 μm to about 138 μm, from about 70 μm to about 137 μm, from about 70 μm to about 136 μm, from about 70 μm to about 135 μm, from about 70 μm to about 134 μm, from about 70 μm to about 133 μm, from about 70 μm to about 132 μm, from about 70 μm to about 131 μm, from about 70 μm to about 130 μm, from about 70 μm to about 129 μm, from about 70 μm to about 128 μm, from about 70 μm to about 127 μm, from about 70 μm to about 126 μm, from about 70 μm to about 125 μm, from about 70 μm to about 124 μm, from about 70 μm to about 123 μm, from about 70 μm to about 122 μm, from about 70 μm to about 121 μm, or from about 70 μm to about 120 μm, such as from about 71 μm to about 120 μm, from about 72 μm to about 120 μm, from about 73 μm to about 120 μm, from about 74 μm to about 120 μm, from about 75 μm to about 120 μm, from about 76 μm to about 120 μm, from about 77 μm to about 120 μm, from about 78 μm to about 120 μm, from about 79 μm to about 120 μm, from about 80 μm to about 120 μm, such as from about 81 μm to about 120 μm, from about 82 μm to about 120 μm, from about 83 μm to about 120 μm, from about 84 μm to about 120 μm, from about 85 μm to about 120 μm, from about 86 μm to about 120 μm, from about 87 μm to about 120 μm, from about 88 μm to about 120 μm, from about 89 μm to about 120 μm, or from about 90 μm to about 120 μm.

According to a 43^{rd} (43) embodiment, in an embodiment of any one of embodiments 32-34, the differentiated epithelial cells may have a thickness of living cell layers of from about 2 μm to about 70 μm, from about 2 μm to about 69 μm, from about 2 μm to about 68 μm, from about 2 μm to about 67 μm, from about 2 μm to about 66 μm, from about 2 μm to about 65 μm, from about 2 μm to about 64 μm, from about 2 μm to about 63 μm, from about 2 μm to about 62 μm, from about 2 μm to about 61 μm, from about 2 μm to about 60 μm, from about 2 μm to about 59 μm, from about 2 μm to about 58 μm, from about 2 μm to about 57 μm, from about 2 μm to about 56 μm, from about 2 μm to about 55 μm, from about 2 μm to about 54 μm, from about 2 μm to about 53 μm, from about 2 μm to about 52 μm, from about 2 μm to about 51 μm, from about 2 μm to about 50 μm, from about 2 μm to about 49 μm, from about 2 μm to about 48 μm, from about 2 μm to about 47 μm, from about 2 μm to about 46 μm, from about 2 μm to about 45 μm, from about 2 μm to about 44 μm, from about 2 μm to about 43 μm, from about 2 μm to about 42 μm, from about 2 μm to about 41 μm, from about 2 μm to about 40 μm, from about 2 μm to about 39 μm, from about 2 μm to about 38 μm, from about 2 μm to about 37 μm, from about 2 μm to about 36 μm, from about 2 μm to about 35 μm, from about 2 μm to about 34 μm, from about 2 μm to about 33 μm, from about 2 μm to about 32 μm, from about 2 μm to about 31 μm, from about 2 μm to about 30 μm, from about 2 μm to about 29 μm, from about 2 μm to about 28 μm, from about 2 μm to about 27 μm, from about 2 μm to about 26 μm, from about 2 μm to about 25 μm, from about 2 μm to about 24 μm, from about 2 μm to about 23 μm, from about 2 μm to about 22 μm, from about 2 μm to about 21 μm, from about 2 μm to about 20 μm, from about 2 μm to about 19 μm, from about 2 μm to about 18 μm, from about 2 μm to about 17 μm, from about 2 μm to about 16 μm, from about 2 μm to about 15 μm, from about 2 μm to about 14 μm, from about 2 μm to about 13 μm, from about 2 μm to about 12 μm, from about 2 μm to about 11 μm, from about 2 μm to about 10 μm, from about 2 μm to about 9 μm, from about 2 μm to about 8 μm, from about 2 μm to about 7 μm, from about 2 μm to about 6 μm, from about 2 μm to about 5 μm, from about 2 μm to about 4 μm, or from about 2 μm to about 3 μm.

According to a 44^{rd} (44) embodiment, in an embodiment of any one of embodiments 32-34, the differentiated epithelial cells may have a thickness of living cell layers of from about 3 μm to about 70 μm, from about 4 μm to about 70 μm, from about 5 μm to about 70 μm, from about 6 μm to about 70 μm, from about 7 μm to about 70 μm, from about 8 μm to about 70 μm, from about 9 μm to about 70 μm, from about 10 μm to about 70 μm, from about 11 μm to about 70 μm, from about 12 μm to about 70 μm, from about 13 μm to about 70 μm, from about 14 μm to about 70 μm, from about 15 μm to about 70 μm, from about 16 μm to about 70 μm, from about 17 μm to about 70 μm, from about 18 μm to about 70 μm, from about 19 μm to about 70 μm, from about 20 μm to about 70 μm, from about 21 μm to about 70 μm, from about 22 μm to about 70 μm, from about 23 μm to about 70 μm, from about 24 μm to about 70 μm, from about 25 μm to about 70 μm, from about 26 μm to about 70 μm, from about 27 μm to about 70 μm, from about 28 μm to about 70 μm, from about 29 μm to about 70 μm, from about 30 μm to about 70 μm, from about 31 μm to about 70 μm, from about 32 μm to about 70 μm, from about 33 μm to about 70 μm, from about 34 μm to about 70 μm, from about 35 μm to about 70 μm, from about 36 μm to about 70 μm, from about 37 μm to about 70 μm, from about 38 μm to about 70 μm, from about 39 μm to about 70 μm, from about 40 μm to about 70 μm, from about 41 μm to about 70 μm, from about 42 μm to about 70 μm, from about 43 μm to about 70 μm, from about 44 μm to about 70 μm, from about 45 μm to about 70 μm, from about 46 μm to about 70 μm, from about 47 μm to about 70 μm, from about 48 μm to about 70 μm, from about 49 μm to about 70 μm, from about 50 μm to about 70 μm, from about 51 μm to about 70 μm, from about 52 μm to about 70 μm, from about 53 μm to about 70 μm, from about 54 μm to about 70 μm, from about 55 μm to about 70 μm, from about 56 μm to about 70 μm, from about 57 μm to about 70 μm, from about 58 μm to about 70 μm, from about 59 μm to about 70 μm, from about 60 μm to about 70 μm, from about 61 μm to about 70 μm, from about 62 μm to about 70 μm, from about 63 μm to about 70 μm, from about 64 μm to about 70 μm, from about 65 μm to about 70 μm, from about 66 μm to about 70 μm, from about 67 μm to about 70 μm, from about 68 μm to about 70 μm, or from about 69 μm to about 70 μm.

According to a 45^{th} (45) embodiment, in an embodiment of any one of embodiments 32-44, the differentiated epithelial cells may be differentiated over predecessor mammalian keratinocytes.

According to a 46^{th} (46) embodiment, in an embodiment of any one of embodiments 32-45, the differentiated epithelial cells may be differentiated over predecessor human keratinocytes.

According to a 47^{th} (47) embodiment, in an embodiment of any one of embodiments 32-46, the differentiated epithelial cells may be cultured for at least 1, 2, 3, 4, 5, 6, 7, 8, 9, 10, 11, 12, 13 14, 15, 16, 17, 18, 19, 20, 21, 22, 23, 24, 25, 26, 27, 28, 29, 30, 31, 32, 33, 34, or 35 days, such as from between 1 to 35 days, 2 to 34 days, 2 to 33 days, 2 to 32 days, 2 to 31 days, 2 to 30 days, 2 to 29 days, 2 to 28 days, 2 to 27 days, 2 to 26 days, 2 to 25 days, 2 to 24 days, 2 to 23 days, 2 to 22 days, 2 to 21 days, 2 to 20 days, 2 to 19 days, 2 to 18 days, 2 to 17 days, 2 to 16 days, 2 to 15 days, 2 to 14 days, 2 to 13 days, 2 to 12 days, 2 to 11 days, 2 to 10 days, 2 to 9 days, 2 to 8 days, 2 to 7 days, 3 to 16 days, 3 to 15 days, 3 to 14 days, 3 to 13 days, 3 to 12 days, 3 to 11 days, 3 to 10 days, 3 to 9 days, 3 to 8 days, 3 to 7 days, 4 to 16 days, 4 to 15 days, 4 to 14 days, 4 to 13 days, 4 to 12 days, 4 to 11 days, 4 to 10 days, 4 to 9 days, 4 to 8 days, 4 to 7 days, 5 to 16 days, 5 to 15 days, 5 to 14 days, 5 to 13 days, 5 to 12 days, 5 to 11 days, 5 to 10 days, 5 to 9 days, 5 to 8 days, or 5 to 7 days.

According to a 48^{th} (48) embodiment, in an embodiment of any one of embodiments 32-47, the differentiated epithelial cells may be cultured on a porous substrate.

According to a 49^{th} (49) embodiment, in an embodiment of any one of embodiments 32-48, the differentiated epithelial cells may be incubated for at least 3 hours.

According to a 50^{th} (50) embodiment, in an embodiment of any one of embodiments 32-49, the differentiated epithelial cells may be incubated at a temperature ranging from about 36° C. to about 38° C.

According to a 51^{st} (51) embodiment, in an embodiment of any one of embodiments 32-50, wherein the differentiated epithelial cells may be incubated at about 4%-6% CO2.

According to a 52^{nd} (52) embodiment, in an embodiment of any one of embodiments 32-51, the differentiated epithelial cells may be incubated at about 40% to about 100% humidity, such as at about 50% to about 99%, such as at about 60% to about 98%, preferably at about 70% to about 97%, preferably at about 45% to about 80%, more preferably at about 50% to about 70%, more preferably at about 55% to about 65%, more preferably at about 92% to about 97%, or more preferably at about 94% to about 96%.

According to a 53^{rd} (53) embodiment, in an embodiment of any one of embodiments 32-52, the conditioned medium comprising the extracellular vesicles may be concentrated, filtered, and/or purified prior to combining the extracellular vesicles with an acceptable carrier to form the composition.

According to a 54^{th} (54) embodiment, in an embodiment of any one of embodiments 32-53, the extracellular vesicles may be isolated from the conditioned media prior to combining the extracellular vesicles with an acceptable carrier to form the composition.

According to a 55^{th} (55) embodiment, in an embodiment of any one of embodiments 32-54, the extracellular vesicles may be lysed prior to combination with an acceptable carrier.

According to a 56^{th} (56) embodiment, in an embodiment of any one of embodiments 32-55, the nutrient medium may be a chemically defined medium.

According to a 57^{th} (57) embodiment, in an embodiment of any one of embodiments 32-56, the differentiated epithelial cells may be cultured in batch culture.

According to a 58^{th} (58) embodiment, in an embodiment of any one of embodiments 32-57, the extracellular vesicles may comprise large extracellular vesicles having a diameter of more than 150 nm and small extracellular vesicles having a diameter 150 nm or less, and the ratio of the small extracellular vesicles to the large extracellular vesicles is no greater than about 30.5:1, no greater than about 30:1, no greater than about 29.5:1, no greater than about 29:1, no greater than about 28.5:1, no greater than about 28:1, no greater than about 27.5:1, no greater than about 27:1, no greater than about 26.5:1, no greater than about 26:1, no greater than about 25.5:1, no greater than about 25:1, no greater than about 24.5:1, no greater than about 24:1, no greater than about 23.5:1, no greater than about 23:1, no greater than about 22.5:1, no greater than about 22:1, no greater than about 21.5:1, no greater than about 21:1, no greater than about 20.5:1, no greater than about 20:1, no greater than about 19.5:1, no greater than about 19:1, no greater than about 18.5:1, no greater than about 18:1, no greater than about 17.5:1, no greater than about 17:1, no greater than about 16.5:1, no greater than about 16:1, no greater than about 15.5:1, no greater than about 15:1, no greater than about 14.5:1, no greater than about 14:1, no greater than about 13.5:1, no greater than about 13:1, no greater than about 12.5:1, no greater than about 12:1, no greater than about 11.5:1, no greater than about 11:1, no greater than about 10.5:1, no greater than about 10:1, no greater than about 9.5:1, no greater than about 9:1, no greater than about 8.5:1, no greater than about 8:1, no greater than about 7.5:1, no greater than about 7:1, no greater than about 6.5:1, no greater than about 6:1, no greater than about 5.5:1, no greater than about 5:1, no greater than about 4.5:1, no greater than about 4:1, no greater than about 3.5:1, no greater than about 3:1, no greater than about 2.5:1, no greater than about 2:1, no greater than about 1.5:1, or no greater than about 1:1 when a) separating the small extracellular vesicles from the conditioned medium, using a combination of tangential flow filtration (TFF) and size-exclusion chromatography (SEC), then b) detecting the number of small extracellular vesicles, and a) separating the large extracellular vesicles from the conditioned medium by TFF, then b) detecting the number of large extracellular vesicles.

According to a 59^{th} (59) embodiment, in an embodiment of any one of embodiments 32-58, the extracellular vesicles may comprise large extracellular vesicles having a diameter of more than 150 nm and small extracellular vesicles having a diameter 150 nm or less, and an average ratio of the small extracellular vesicles to the large extracellular vesicles may range from about 1:1 to about 30.5:1, from about 1:1 to about 30:1, from about 1:1 to about 29.5:1, from about 1:1 to about 29:1, from about 1:1 to about 28.5:1, from about 1:1 to about 28:1, from about 1:1 to about 27.5:1, from about 1:1 to about 27:1, from about 1:1 to about 26.5:1, from about 1:1 to about 26:1, from about 1:1 to about 25.5:1, from about 1:1 to about 25:1, from about 1:1 to about 24.5:1, from about 1:1 to about 24:1, from about 1:1 to about 23.5:1, from about 1:1 to about 23:1, from about 1:1 to about 22.5, from about 1:1 to about 22:1, from about 1:1 to about 21.5:1, from about 1:1 to about 21:1, from about 1:1 to about 20:1, from about 1:1 to about 19:1, about from 1:1 to about 18:1, from about 1:1 to about 17:1, from about 1:1 to about 16:1, from about 1:1 to about 15:1, from about 1:1 to about 14:1, from about 1:1 to about 13:1, from about 1:1 to about 12:1, from about 1:1 to about 11:1, from about 1:1 to about 10:1, from about 1:1 to about 9:1, from about 1:1 to about 8:1, from about 1:1 to about 7:1, from about 1:1 to about 6:1, from about 1:1 to about 5:1, from about 1:1 to about 4:1, from about 1:1 to about 3:1, from about 1:1 to about 2:1, from about 2:1 to about 20:1, from about 2:1 to about 19:1, from about 2:1 to about 18:1, from about 2:1 to about 17:1, from about 2:1 to about 16:1, from about 2:1 to about 15:1, from about 2:1 to about 14:1, from about 2:1 to about 13:1, from about 2:1 to about 12:1, from about 2:1 to about 11:1, from about 2:1 to about 10:1, from about 2:1 to about 9:1, from about 2:1 to about 8:1, from about 2:1 to about 7:1, from about 2:1 to about 6:1, from about 2:1 to about 5:1, from about 2:1 to about 4:1, from about 2:1 to about 3:1, from about 3:1 to about 20:1, from about 3:1 to about 19:1, from about 3:1 to about 18:1, from about 3:1 to about 17:1, from about 3:1 to about 16:1, from about 3:1 to about 15:1, from about 3:1 to about 14:1, from about 3:1 to about 13:1, from about 3:1 to about 12:1, from about 3:1 to about 11:1, from about 3:1 to about 10:1, from about 3:1 to about 9:1, from about 3:1 to about 8:1, from about 3:1 to about 7:1, from about 3:1 to about 6:1, from about 3:1 to about 5:1, or from about 3:1 to about 4:1 when a) separating the small extracellular vesicles from the conditioned medium, using a combination of tangential flow filtration (TFF) and size-exclusion chromatography (SEC), then b) detecting the number of small extracellular vesicles, and a) separating the large extracellular vesicles from the conditioned medium by TFF, then b) detecting the number of large extracellular vesicles.

According to a 60^{th} (60) embodiment, in an embodiment of any one of embodiments 32-59, the composition may comprise a population of CD9 positive extracellular vesicles, wherein a ratio of CD63 to CD81 detected in the population ranges from at least about 0.2:1, from at least about 0.3:1, from at least about 0.4:1, from at least about 0.5:1, from at least about 0.6:1, from at least about 0.7:1, from at least about 0.8:1, from at least about 0.9:1, or from at least about 1:1, or from at least about 1.1:, or from at least about 1.2:1, or from at least about 1.3:1, or from at least about 1.4:1, or from at least about 1.5:1, or from at least about 1.6:1, or from at least about 1.7:1, or from at least about 1.9:1, or from at least about 2:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

According to a 61^{st} (61) embodiment, in an embodiment of any one of embodiments 32-60, the composition may comprise a population of CD9 positive extracellular vesicles wherein the ratio of CD63 to CD81 detected in the population ranges from about 0.2:1 to about 2.8:1, from about 0.2:1 to about 2.7:1, from about 0.3:1 to about 2.6:1, from about 0.3:1 to about 2.5:1, from about 0.4:1 to about 2.4:1, from about 0.4:1 to about 2.3:1, from about 0.5:1 to about 2.2:1, from about 0.6:1 to about 2.1:1, from about 0.6:1 to about 2.0:1, from about 0.6:1 to about 1.9:1, from about 0.7:1 to about 1.8:1, from about 0.7:1 to about 1.7:1, from about 0.8:1 to about 1.6:1, from about 0.8:1 to about 1.5:1, from about 0.9:1 to about 1.4:1, or from about 0.9:1 to about 1.3:1 when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

According to a 62^{nd} (62) embodiment, in an embodiment of any one of embodiments 32-61, the composition may further comprise small molecules, biologics, therapeutic agents, preservatives, and/or enzymes.

A 63^{rd} (63) embodiment comprises a composition comprising a cosmetic carrier and a population of CD9 positive extracellular vesicles wherein the ratio of CD63 to CD81 detected in the population ranges from at least about 0.2:1, from at least about 0.3:1, from at least about 0.4:1, from at least about 0.5:1, from at least about 0.6:1, from at least about 0.7:1, from at least about 0.8:1, from at least about 0.9:1, or from at least about 1:1, or from at least about 1.1:, or from at least about 1.2:1, or from at least about 1.3:1, or from at least about 1.4:1, or from at least about 1.5:1, or from at least about 1.6:1, or from at least about 1.7:1, or from at least about 1.9:1, or from at least about 2:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

A 64^{th} (64) embodiment comprises a composition comprising cosmetic carrier and a population of CD9 positive extracellular vesicles wherein the ratio of CD63 to CD81 detected in the population ranges from about 0.2:1 to about 2.8:1, from about 0.2:1 to about 2.7:1, from about 0.3:1 to about 2.6:1, from about 0.3:1 to about 2.5:1, from about 0.4:1 to about 2.4:1, from about 0.4:1 to about 2.3:1, from about 0.5:1 to about 2.2:1, from about 0.6:1 to about 2.1:1, from about 0.6:1 to about 2.0:1, from about 0.6:1 to about 1.9:1, from about 0.7:1 to about 1.8:1, from about 0.7:1 to about 1.7:1, from about 0.8:1 to about 1.6:1, from about 0.8:1 to about 1.5:1, from about 0.9:1 to about 1.4:1, or from about 0.9:1 to about 1.3:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

According to a 65^{th} (65) embodiment, in an embodiment of embodiments 63 or 64, the composition may further comprise small molecules, biologics, therapeutic agents, preservatives, and/or enzymes.

A 66^{th} (66) embodiment comprises a method of making a composition, wherein the composition comprises population of CD9 positive extracellular vesicles wherein the ratio of CD63 to CD81 detected in the population ranges from at least about 0.2:1, from at least about 0.3:1, from at least about 0.4:1, from at least about 0.5:1, from at least about 0.6:1, from at least about 0.7:1, from at least about 0.8:1, from at least about 0.9:1, or from at least about 1:1, or from at least about 1.1:, or from at least about 1.2:1, or from at least about 1.3:1, or from at least about 1.4:1, or from at least about 1.5:1, or from at least about 1.6:1, or from at least about 1.7:1, or from at least about 1.9:1, or from at least about 2:1 when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

A 67^{th} (67) embodiment comprises a method of making a composition, wherein the composition comprises population of CD9 positive extracellular vesicles wherein the ratio of CD63 to CD81 detected in the population from about 0.2:1 to about 2.8:1, from about 0.2:1 to about 2.7:1, from about 0.3:1 to about 2.6:1, from about 0.3:1 to about 2.5:1, from about 0.4:1 to about 2.4:1, from about 0.4:1 to about 2.3:1, from about 0.5:1 to about 2.2:1, from about 0.6:1 to about 2.1:1, from about 0.6:1 to about 2.0:1, from about 0.6:1 to about 1.9:1, from about 0.7:1 to about 1.8:1, from about 0.7:1 to about 1.7:1, from about 0.8:1 to about 1.6:1, from about 0.8:1 to about 1.5:1, from about 0.9:1 to about 1.4:1, or from about 0.9:1 to about 1.3:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

According to a 68^{th} (68) embodiment, in an embodiment of embodiments 66 or 67, the composition further comprises small molecules, biologics, therapeutic agents, preservatives, and/or enzymes.

A 69^{th} (69) embodiment comprises a method of treatment comprising applying the composition of any one of embodiments 1-33, 63-65, or 80 to skin.

A 70^{th} (70) embodiment comprises a method of growing epidermal cells comprising applying the composition of any one of embodiments 1-33, 63-65, or 80 to an epidermis.

A 71^{st} (71) embodiment comprises a method for promoting fibroblast growth comprising applying the composition of embodiments 11-33, 63-65, or 80 to skin.

A 72^{nd} (72) embodiment comprises a method for increasing keratinocyte growth factor release from skin cells comprising applying the composition of embodiments 1-33, 63-65, or 80 to skin.

A 73^{rd} (73) embodiment comprises a method for inducing keratinocyte growth factor release from skin cells comprising applying the composition of any one of embodiments 1-33, 63-65 or 80, to skin.

A 74^{th} (74) embodiment comprises a use of the compositions of any one of embodiments 1-33, 63-65, or 80 as a cosmetic.

A 75^{th} (75) embodiment comprises a use of the compositions of any one of embodiments 1-33, 63-65, or 80 as a medicament.

A 76^{th} (76) embodiment comprises a method of use comprising applying the composition of any one of embodiments 1-33, 63-65, or 80 to skin.

A 77^{th} (77) embodiment comprises the compositions of any one of embodiments 1-33, 63-65, or 80 for use in a therapeutic.

A 78^{th} (78) embodiment comprises the compositions of any one of embodiments 1-33, 63-65, or 80 for use in a medicament in the treatment of a skin condition.

A 79^{th} (79) embodiment comprises the use of the compositions of any one of embodiments 1-33, 63-65, or 80 in the manufacture of a medicament for the treatment of a skin condition.

An 80^{th} (80) embodiment comprises a composition comprising differentiated epithelial cell(s) collected conditioned medium-derived extracellular vesicles and an acceptable cosmetic carrier, wherein the differentiated epithelial cell(s) are cultured at an air-liquid interface in a nutrient medium sufficient to meet the nutritional needs required to grow the cells in vitro to form differentiated epithelial cells.

According to an 81^{st} (81) embodiment, in an embodiment of any one of embodiments 1-33, 63-65, 76, 77, or 80, the differentiated epithelial cell(s) are cultured at an air-liquid interface in a nutrient medium sufficient to meet the nutritional needs required to grow the cells in vitro to form partially differentiated epithelial cells.

According to an 82^{nd} (82) embodiment, in an embodiment of any one of embodiments 1-33, 63-65, 76, 77, or 80, the differentiated epithelial cell(s) may be cultured at an air-liquid interface in a nutrient medium sufficient to meet the nutritional needs required to grow the cells in vitro to form fully differentiated epithelial cell(s).

According to an 83^{rd} (83) embodiment, in an embodiment of any one of embodiments 32-62, 66-72, or 75, the differentiated epithelial cell(s) may be cultured at an air-liquid interface in a nutrient medium sufficient to meet the nutritional needs required to grow the cells in vitro to form partially differentiated epithelial cell(s).

According to an 85^{th} (85) embodiment, in an embodiment of any one of embodiments 32-62, 66-72, or 75, wherein the differentiated epithelial cell(s) may be cultured at an air-liquid interface in a nutrient medium sufficient to meet the nutritional needs required to grow the cells in vitro to form fully differentiated epithelial cell(s).

According to an 86^{th} (86) embodiment, in an embodiment of any one of embodiments 73, 74, or 78, the differentiated epithelial cells may be cultured at an air-liquid interface in a nutrient medium sufficient to meet the nutritional needs required to grow the cells in vitro to form partially differentiated epithelial cell(s).

According to an 84^{th} (84) embodiment, in an embodiment of any one of embodiments, the differentiated epithelial cells may be cultured at an air-liquid interface in a nutrient medium sufficient to meet the nutritional needs required to grow the cells in vitro to form fully differentiated epithelial cells.

In this application, the use of the singular includes the plural unless specifically stated otherwise. Thus, the terms “a,” “an,” and “the” are understood to encompass the plural as well as the singular. In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one unit unless specifically stated otherwise.

The term “and/or” should be understood to include both the conjunctive and the disjunctive. For example, “preservatives and/or enzymes” means “preservatives and enzymes” as well as “preservatives or enzymes,” and expressly covers instances of either without reference to the other.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%— 11% and “about 2%” means 1.8%-2.2%), such as within 9%, 8%, 7%, 6%, 5%, 4%, 3%, 2%, or 1%, according to various embodiments. All numbers (including ratios, concentrations, etc.) disclosed herein are understood to include the term “about” whether or not present.

As used herein, the expressions “ranging from” and “between” are inclusive of the endpoints of the recited range(s).

As used herein, all ranges provided are meant to include every specific range within, and combination of sub ranges between, the given ranges. Thus, a range from 1-5, includes specifically 1, 2, 3, 4, and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “comprising” (and its grammatical variations) as used herein is used in the inclusive sense of “having” or “including” and not in the exclusive sense of “consisting only of.”

The term “substantially free” or “essentially free” as used herein means that there is less than about 5% by weight of a specific material added to a composition, based on the total weight of the compositions. The compositions of the disclosure may be free of the components or may be “substantially free” or “essentially free” of the components described for optional inclusion in said compositions. Nonetheless, the compositions may include less than about 4%, less than about 3%, less than about 2%, less about 1%, less than about 0.5%, less than about 0.1%, less than about 0.01%, less than about 0.001%, or none of the specified material or components. Likewise, as used herein, the term “avoid” or “avoiding” the inclusion of a component means that there is less than about 5% by weight of a specific material added to a composition, based on the total weight of the compositions, such as less than about 4%, less than about 3%, less than about 2%, less about 1%, less than about 0.5%, less than about 0.1%, less than about 0.01%, less than about 0.001%, or none of the specified material or components.

Unless otherwise expressly stated, it is in no way intended that any method set forth herein be construed as requiring that its steps be performed in a specific order. Accordingly, where a method claim does not expressly recite an order to be followed by its steps or it is not otherwise specifically stated in the claims or descriptions that the steps are to be limited to a specific order, it is no way intended that any particular order be inferred.

The compositions and methods of the present disclosure can comprise, consist of, or consist essentially of the essential elements and limitations of the disclosure described herein, as well as any additional or optional ingredients, components, or limitations described herein or otherwise useful.

All references disclosed herein are incorporated by reference in their entireties.

Several embodiments having been described above, may be better understood by reference to examples. The following examples are intended for illustration purposes only, and should not be construed as limiting the scope of the invention in any way.

### Examples

The following examples are intended to be non-limiting and explanatory in nature only.

### Example 1: Preparation of Conditioned Media

SkinEthic™ RHE/Reconstructed Human Epidermis (referred to as “RHE” in the Examples) was grown on polycarbonate filters in culture media at the air-liquid interface. A subset of tissues were processed on days 3, 6, and 17, at which point conditioned media was collected from the culture and tissues were fixed and stained using hematoxylin and eosin (H&E) stain. The thickness of living cell layers was measured on days 3, 6, and 17 and is summarized in Table 1 below. A granular layer was detected on day 17, but was absent on days 3 and 6.

TABLE 1
Thickness of Living Cell Layers of Reconstructed
Human Epidermis

| | Thickness (μm) | | |
|---|---|---|---|
| | Min | Max | Granular Layer |
| RHE— | 2.88 | 15 | Absence |
| Day 3 | | | |
| RHE— | 18.1 | 55.3 | Absence |
| Day 6 | | | |
| RHE— | 90.3 | 117 | Presence |
| Day 17 | | | |

The collected conditioned media samples from days 3, 6, and 17 were frozen, thawed and analyzed using the methods described in the Examples below to evaluate differences in composition and/or contents at each disclosed timepoint.

### Example 2: Identification and Quantitation of Extracellular Vesicles and Proteins

Conditioned media was thawed, centrifuged, and analyzed for the concentration and size distribution of particles consistent with small extracellular vesicles (“Small EVs”) and large extracellular vesicles (“Large EVs”) using nanoparticle tracking analysis (NTA) for direct, real-time visualization and analysis of nanoparticles in liquids. The quantification was performed (1) on conditioned media (Table 2) and (2) on recovered liquid fractions after isolation and concentration processes for large and small extracellular vesicles (data not shown). Results are disclosed in Table 2 below.

TABLE 2
Particle content of conditioned media collected from 2D
keratinocytes and 3D reconstructed human epidermis at various time points

| | | Batch 1 | | | Batch 2 | | |
|---|---|---|---|---|---|---|---|
| Raw | | | Quantity | | | Quantity | |
| material | | Concentration | of particles | Median | Concentration | of particles | Median |
| 2D | EV | (parts/mL) | per culture | size | (parts/mL) | per culture | size |
| Keratinocytes | subtypes | per tissue | area (cm³) | (nm) | per tissue | area (cm³) | (nm) |
| 2D | Large | N/A | 1.02 E⁺⁰⁷ | 168.1 | N/A | 3.93 E⁺⁰⁶ | 179.4 |
| Keratinocytes | Small | N/A | 8.65 E⁺⁰⁷ | 99.5 | N/A | 6.76 E⁺⁰⁷ | 94.9 |
| RHE-Day 3 | Large | 2.19 E⁺⁰⁶ | 4.38 E⁺⁰⁸ | 255 | 1.25 E⁺⁰⁵ | 2.50 E⁺⁰⁷ | 198.4 |
| | Small | 6.25 E⁺⁰⁶ | 1.25 E⁺⁰⁹ | 89.6 | 6.56 E⁺⁰⁵ | 1.31 E⁺⁰⁸ | 86.2 |
| RHE-Day 6 | Large | 5.94 E⁺⁰⁵ | 1.19 E⁺⁰⁸ | 217.8 | 2.29 E⁺⁰⁵ | 4.58 E⁺⁰⁷ | 170 |
| | Small | 3.75 E⁺⁰⁶ | 7.50 E⁺⁰⁸ | 88.9 | 9.90 E⁺⁰⁵ | 1.98 E⁺⁰⁸ | 79.2 |
| RHE-Day 17 | Large | 8.13 E⁺⁰⁵ | 1.63 E⁺⁰⁸ | 119.8 | 1.98 E⁺⁰⁵ | 3.96 E⁺⁰⁷ | 243.3 |
| | Small | 9.38 E⁺⁰⁶ | 1.88 E⁺⁰⁹ | 81.6 | 1.25 E⁺⁰⁶ | 2.50 E⁺⁰⁸ | 67.9 |

As demonstrated above in Table 2, particles consistent with small and large extracellular vesicles were identified in conditioned media from differentiated epithelial cells on days 3, 6, and 17 of cell culture.

Particles consistent with small EVs 150 nm) and large EVs (>150 nm) were isolated and concentrated from samples of conditioned media using tangential flow filtration and size-exclusion chromatography. The small extracellular vesicles are separated out using tangential flow filtration. The TFF-separated small extracellular vesicles were then concentrated and purified using size-exclusion chromatography. The large extracellular vesicles were separated out using tangential flow filtration.

### Example 3: Identification and Quantitation of Extracellular Vesicle Surface Markers

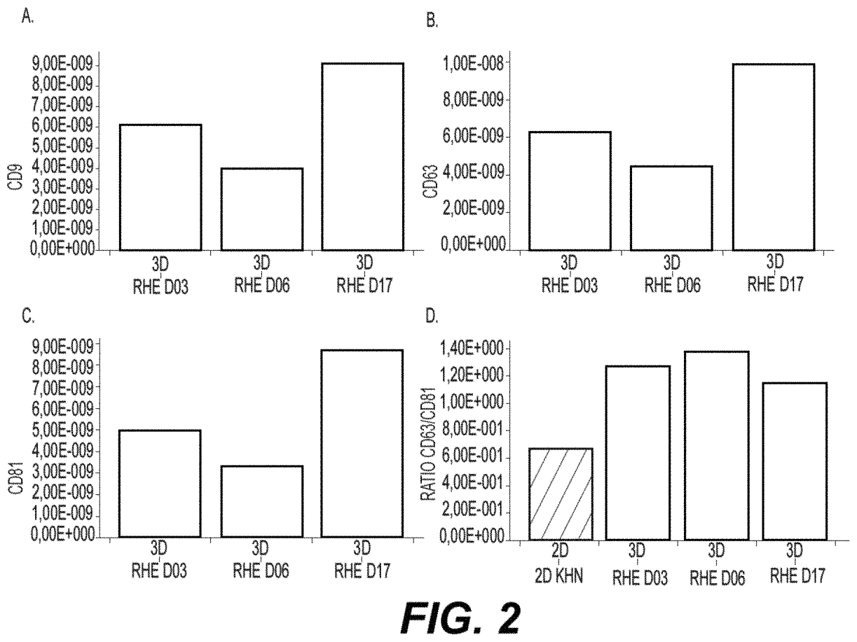

Particles in the isolated small and large EV fractions were tested for the presence of tetraspanins CD9, CD63, and CD81, which serve as common markers for EVs, using enzyme-linked immunosorbent assays (ELISAs). The relative expression of CD81 and CD63 tetraspanins was determined on CD9-positive small extracellular vesicles using a sandwich enzyme-linked immunosorbent assay (Hansabiomed Life Sciences). Briefly, the plate was coated with mouse anti-human CD9 antibody and incubated with isolated small or large extracellular vesicles. Vesicles were captured using the CD9 antibody and were quantified using an anti-human CD63 or CD81 biotinylated antibody. The detection of CD63+/CD9+ vesicles or CD81+/CD9+ vesicles were performed using HRP-streptavidin (Biorad). Normalized results disclosed throughout are expressed as a ratio of the presence of the expressed biomarker (e.g. tetraspanins) to background noise, and normalized by the number of particles in the ELISA assay. Thus, biomarker expression is related to a ratio of the biomarker's absorbance value to the background signal. Tetraspanin's fold increase values were normalized to the recovered extracellular particle count to approximate the relative number of tetraspanins per extracellular vesicle. All extracellular vesicle samples demonstrated the presence of tetraspanins compared to background, suggesting the presence of EVs in the small EV fraction regardless of culture conditions. When compared to conditioned media from 2D keratinocyte culture, there was an increased ratio of CD63+/CD81+ relative concentrations among CD9+ small EVs, suggesting a different expression pattern of common EV markers when comparing EVs collected from 3D RHE culture to those from keratinocytes in 2D. See FIG. 2 (D).

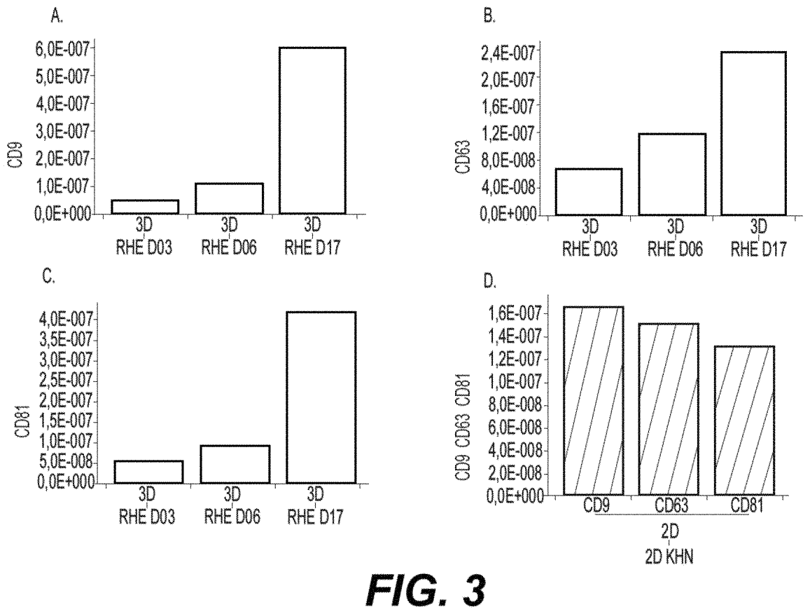

Particles in the large EV fraction were also analyzed for tetraspanins (ELISA) to determine if EVs were present within the sample. Similar to small EVs, tetraspanins were detected in the large EV fraction as well. See FIG. 3 (A-D).

### Example 4: Identification and Quantitation of Internal Extracellular Vesicle Biomarkers

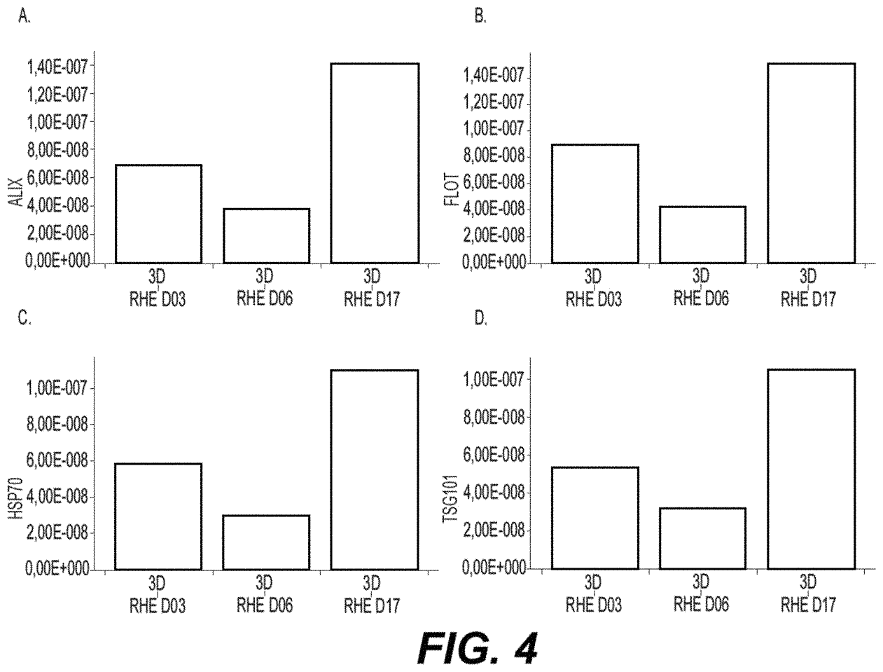

Small EVs were lysed and analyzed by enzyme-linked immunosorbent assays (ELISAs) for the presence of common internal markers of EVs: ALG-2-interacting protein X (ALIX), tumor susceptibility gene 101 (TSG101), flotillin-1 (FLOT), and heat shock protein 70 (HSP70). See FIG. 4 (A-D). All small EV samples contained ALIX, TSG101, FLOT and HSP70. Internal marker fold increase values were normalized to the recovered particle count to approximate the relative level of internal markers per extracellular vesicle.

Small EVs collected from 2D keratinocyte cultures were also lysed and the lysate was analyzed by enzyme-linked immunosorbent assays which demonstrated the presence of internal markers ALIX, FLOT, HSP70, and TSG101 (data not shown).

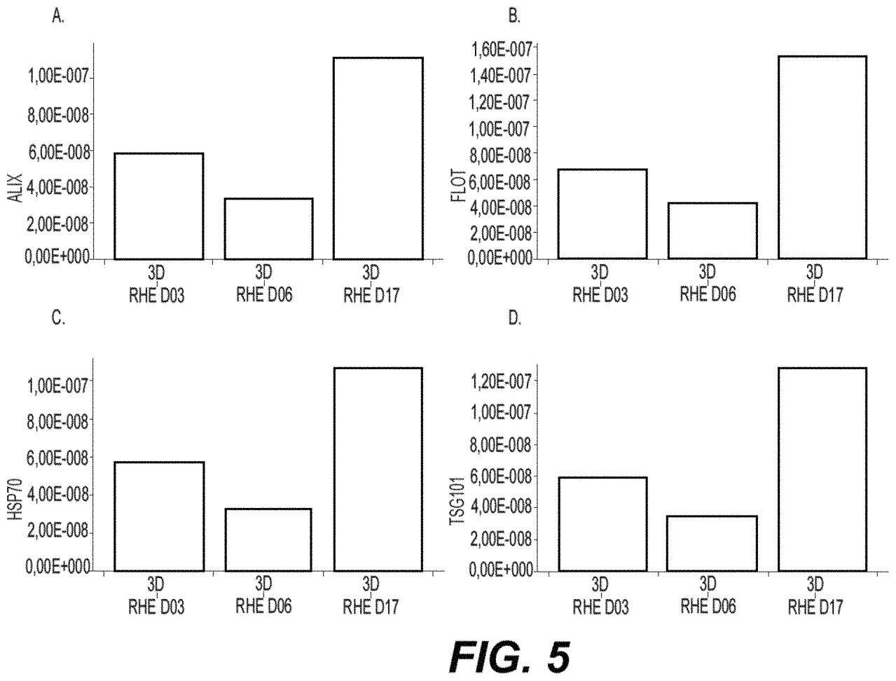

Large EVs were also lysed and analyzed by ELISA for the presence of internal markers ALIX, TSG101, FLOT, and HSP70. See FIG. 5 (A-D). All extracellular vesicle samples contained ALIX, TSG101, FLOT and HSP70. Internal marker fold increase values were normalized to the recovered particle count to approximate relative internal markers per extracellular vesicle. Large EVs from 2D keratinocyte conditioned media also exhibited the presence of ALIX, TSG101, FLOT and HSP70 (data not shown).

### Example 5: Quantitation of Fibroblast Proliferation after Exposure to Small EVs

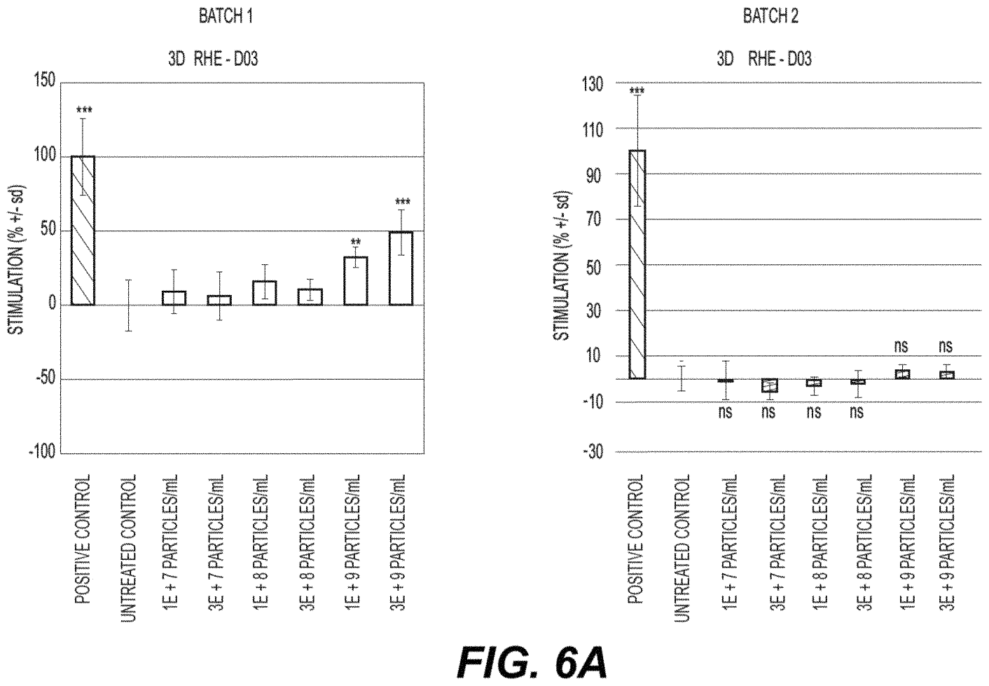

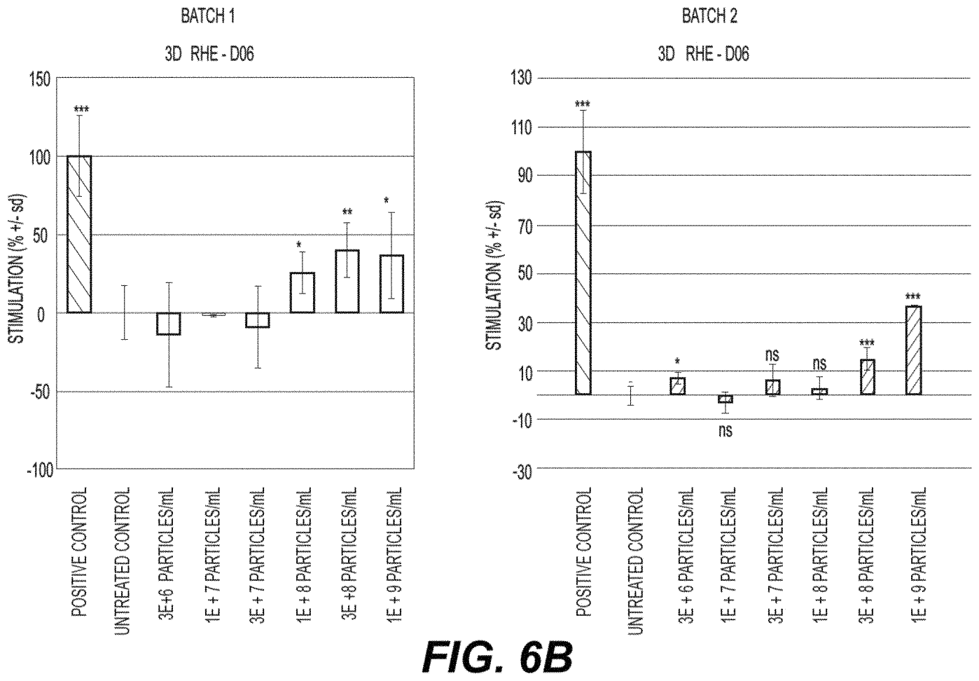

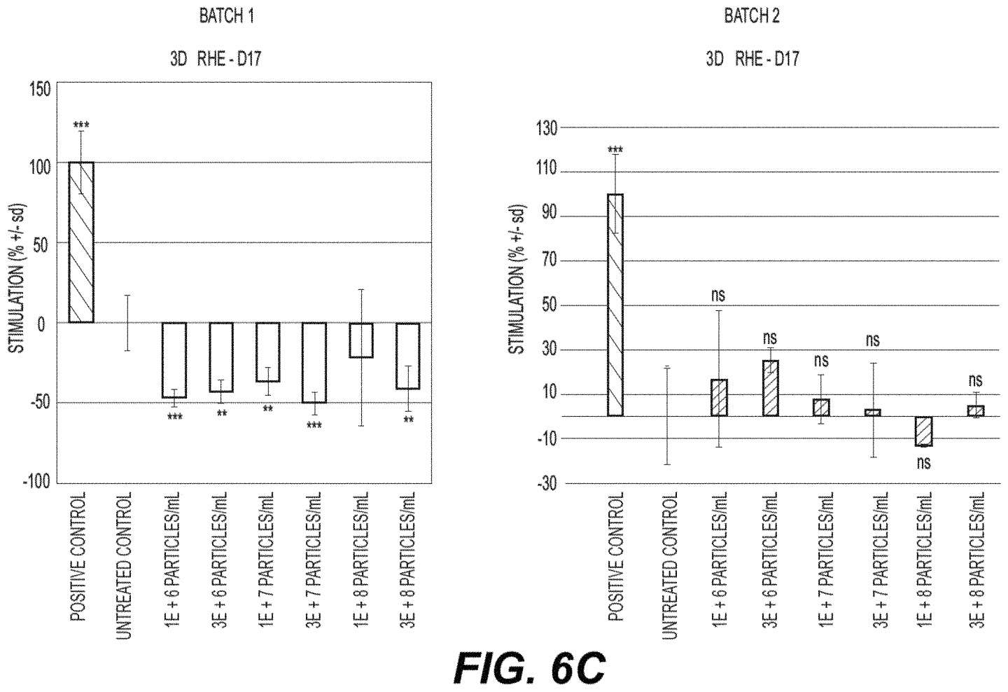

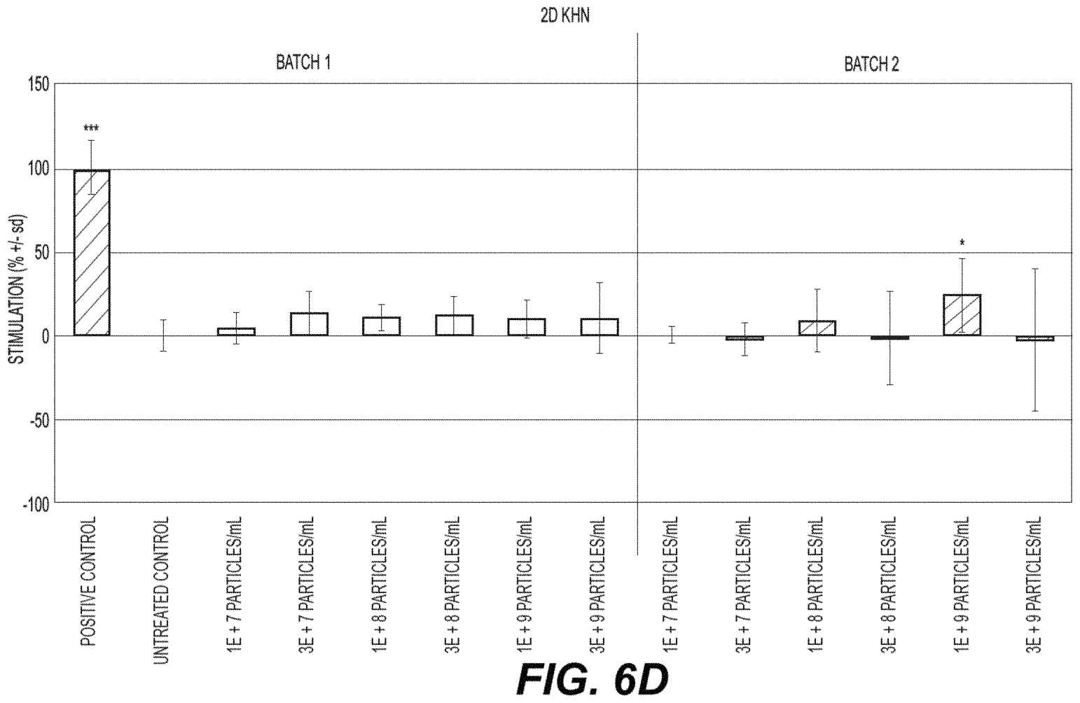

Small EVs isolated from differentiated epithelial cell conditioned media were tested for their ability to stimulate fibroblast proliferation. Briefly, small EVs were added at increasing concentrations to normal human dermal fibroblasts seeded in two-dimensional culture plates. After 72 hours in culture, proliferation was quantified using a CyQUANT™ Cell Proliferation Assay (Thermo Fisher). Complete culture medium was used as a positive control. Percent increases in cell growth over normal, or the stimulation percentage, after exposure to small extracellular vesicles from two batches of RHE conditioned media on days 3, 6 and 17 of cell culture respectively and two batches of two-dimensional keratinocyte cultures is disclosed in FIGS. 6A-6D.

### Example 6: Quantitation of Keratinocyte Growth Factor (KGF) Secretion after Exposure to Small EVs

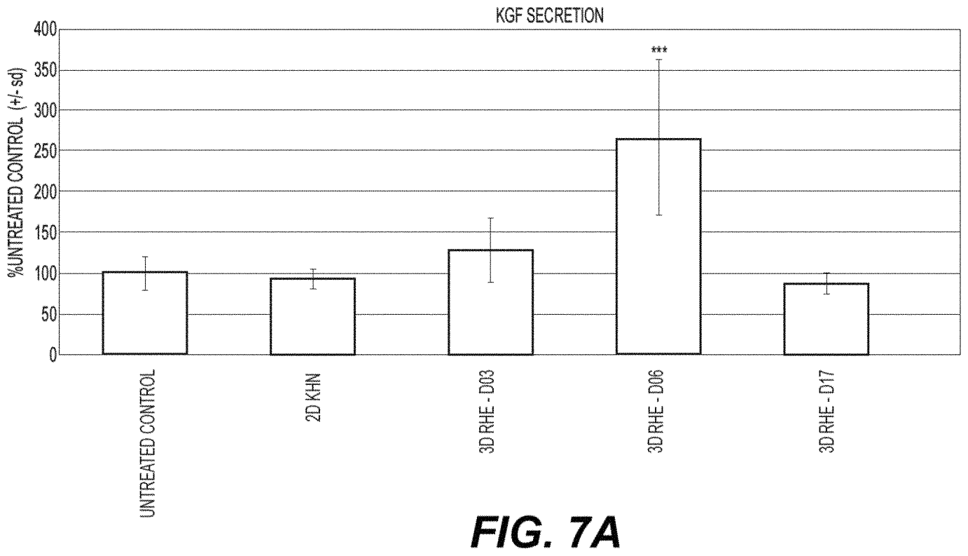

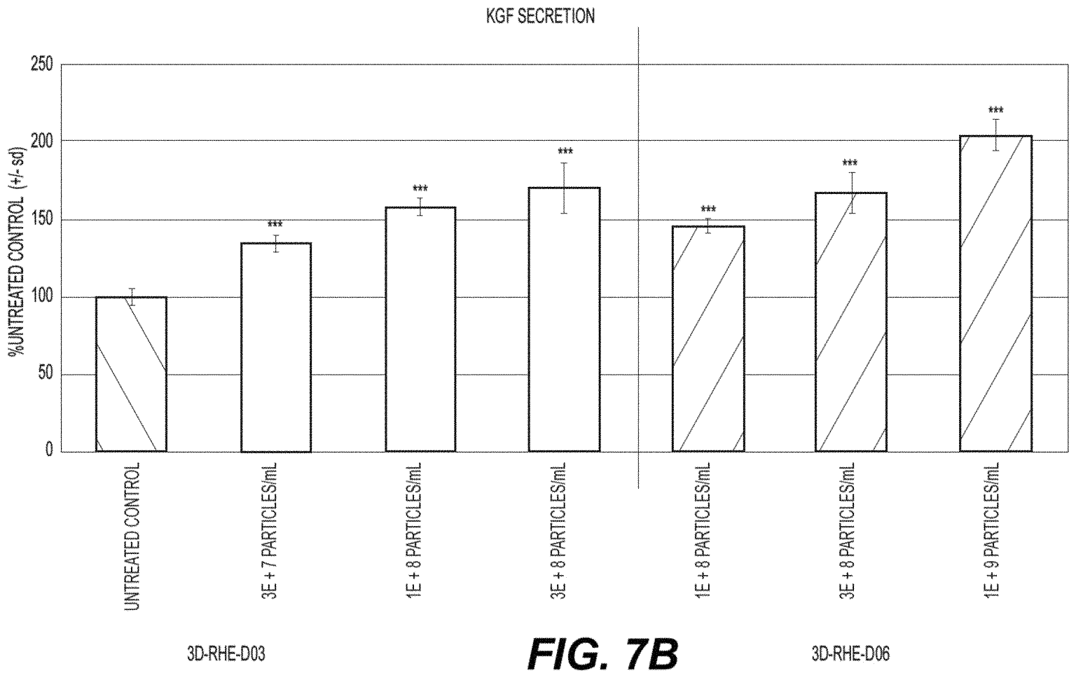

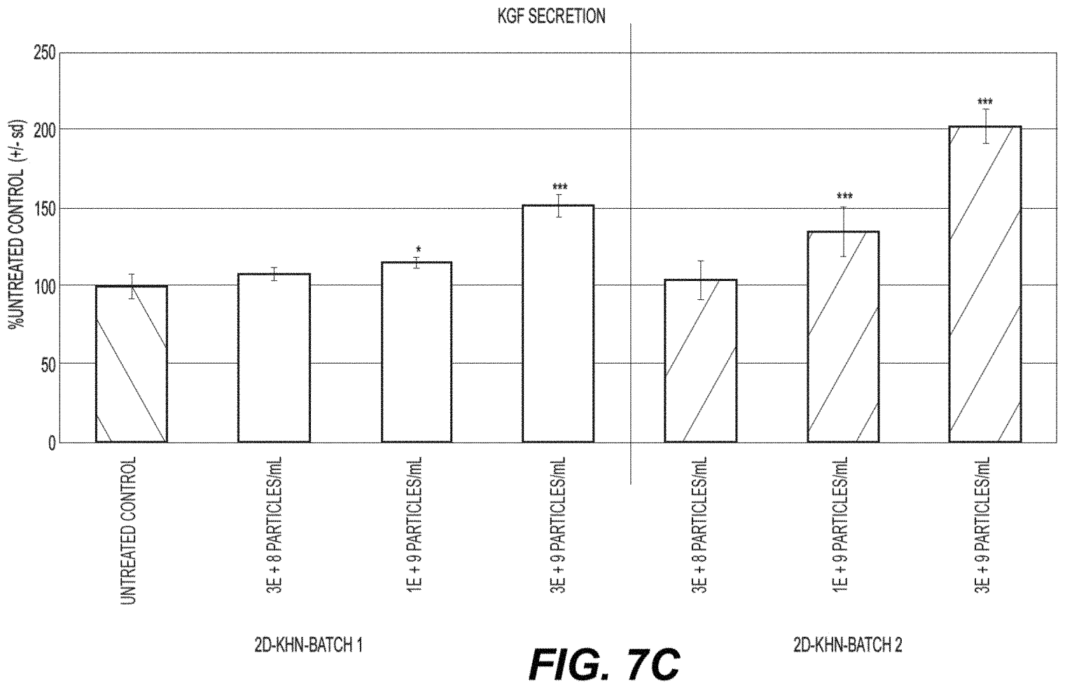

Small EVs were also tested for their ability to stimulate keratinocyte growth factor (KGF) secretion from fibroblasts. Small EVs were added to normal human dermal fibroblasts seeded in two-dimensional culture plates. After 72 hours in culture, conditioned media was collected and the concentration of KGF in the media was determined using ELISA (R&D Systems). Fibroblasts demonstrated a concentration-dependent increase in KGF secretion after exposure to small EVs from day 3 and day 6 RHE conditioned media. See FIGS. 7A-7C.

1. A composition comprising extracellular vesicles and a carrier,
wherein the extracellular vesicles are derived from a conditioned medium collected from differentiated epithelial cells, cultured at an air-liquid interface in a nutrient medium sufficient to meet the nutritional needs required to grow the cells in vitro to form the differentiated epithelial cells.

2. The composition of claim 1, wherein the differentiated epithelial cells were cultured at an air-liquid interface in a nutrient medium sufficient to meet the nutritional needs required to grow the cells in vitro to form partially differentiated epithelial cells.

3. The composition of claim 1, wherein the differentiated epithelial cells comprise on average at least 2 living cell layers.

4. The composition of claim 1, wherein the differentiated epithelial cells comprise on average 1 to 8 living cell layers.

5. The composition of claim 1, wherein the differentiated epithelial cells form one or more living cell layers comprising a basal layer, a stratum spinosum, a granular layer, and/or a stratum corneum.

6. The composition of claim 1, wherein the differentiated epithelial cells comprise a thickness of living cell layers of at least about 2 μm.

7. The composition of claim 1, wherein the differentiated epithelial cells comprise a thickness of living cell layers of about 2 μm to about 141 μm.

8. The composition of claim 1, wherein the differentiated epithelial cells have differentiated over predecessor human keratinocytes.

9. The composition of claim 1, wherein the differentiated epithelial cells were cultured for at least 1 day.

10. The composition of claim 1, wherein the differentiated epithelial cells were cultured for 1 to 35 days.

11. The composition of claim 1, wherein the differentiated epithelial cells were cultured on a porous substrate in a chemically defined medium for at least about 3 hours and incubated at a temperature ranging from about 36° C. to about 38° C., at about 4%-6% CO2, and at about 40% to about 100% humidity.

12. The composition of claim 1, wherein the conditioned medium comprising the extracellular vesicles was concentrated, filtered, and/or purified prior to combining the extracellular vesicles with the carrier.

13. The composition of claim 1, wherein the extracellular vesicles were isolated from the conditioned media prior to combining the extracellular vesicles with the carrier.

14. The composition of claim 1, wherein the extracellular vesicles were lysed prior to combination with the carrier.

15. The composition of claim 1, comprising a population of CD9 positive extracellular vesicles, when the ratio of CD63 to CD81 detected in the population is at least about 0.2:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

16. The composition of claim 1, comprising a population of CD9 positive extracellular vesicles wherein the ratio of CD63 to CD81 detected in the population ranging from about 0.2:1 to about 2.8:1, when CD63 and CD81 are detected on small extracellular vesicles comprising CD9 and having a diameter of 150 nm or less using enzyme-linked immunosorbent assays, when CD9 is captured by enzyme-linked immunosorbent assays after the vesicles are isolated from the conditioned medium using a) tangential flow filtration then b) size-exclusion chromatography, and when the relative concentrations of CD63 and CD81 are determined using enzyme-linked immunosorbent assays.

17. The composition of claim 1, wherein the composition further comprises at least one additional component chosen from small molecules, biologics, therapeutic agents, preservatives, and/or enzymes.

18. A method of making a composition comprising combining extracellular vesicles and a carrier,
wherein the extracellular vesicles are derived from a conditioned medium collected from differentiated epithelial cells cultured at an air-liquid interface in a nutrient medium sufficient to meet the nutritional needs required to grow the cells in vitro to form the differentiated epithelial cells.

19. A method of treatment comprising applying a composition comprising extracellular vesicles to skin,
wherein the extracellular vesicles are derived from a conditioned medium collected from differentiated epithelial cells cultured at an air-liquid interface in a nutrient medium sufficient to meet the nutritional needs required to grow the cells in vitro to form the differentiated epithelial cells.

20. The method of claim 19, wherein the treatment is a cosmetic treatment and/or a medical treatment.
